(19) United States
(12) Patent Application Publication
(10) Pub. No.: US 2025/0352111 A1
(43) Pub. Date: Nov. 20, 2025

(54) Systems and Methods for Body Surface Colonic Mapping

(71) Applicant: Alimetry Limited, Auckland (NZ)

(72) Inventors: Jonathan Christopher Erickson, Lexington, VA (US); Armen A. Gharibans, Auckland (NZ); Gregory B. O'Grady, Auckland (NZ); Ho Beom Seo, Calgary (CA)

(73) Assignee: Alimetry Limited, Auckland (NZ)

(21) Appl. No.: 18/933,661

(22) Filed: Oct. 31, 2024

Related U.S. Application Data
(60) Provisional application No. 63/648,594, filed on May 16, 2024.

(51) Int. Cl. A61B 5/279; A61B 5/00

(52) U.S. Cl. CPC A61B 5/279; A61B 5/42; A61B 5/6833; A61B 5/726

(57) ABSTRACT

Embodiments of the present disclosure provide methods and systems for mapping colonic and rectal activity with an electrode array patch disposed over an abdomen skin surface of a patient. The method includes non-invasively measuring electrical signals associated with colonic activity of the patient with the electrode array patch over a predetermined time period, receiving the measured electrical signals from the electrode array patch over the predetermined time period, determining one or more abnormal cyclic motor patterns (CMPs) and/or abnormal high amplitude propagated contractions (HAPCs) based at least in part on the received measured electrical signals, and generating a report comprising at least the determination of the one or more abnormal CMPs and/or abnormal HAPCs.

### CROSS-REFERENCES TO RELATED APPLICATIONS

The present application claims the benefit of priority of U.S. Provisional Application No. 63/648,594 filed May 16, 2024, entitled, “SYSTEMS AND METHODS FOR BODY SURFACE COLONIC MAPPING,” which is incorporated herein by reference in its entirety for all purposes.

### BACKGROUND

Functional disorders of the colon, including irritable bowel syndrome, affect an estimated ≈10% of the population worldwide, with the highest prevalence in the United States and Europe. Symptoms of abdominal pain and altered bowel habits can be debilitating and result in a profound negative impact on quality of life. Abnormal colonic motility has been implicated in many other significant conditions, including chronic constipation, fecal incontinence, postoperative ileus, and colonic pseudo-obstruction. Such diseases may share similar features, but they have distinct underlying mechanisms, complicating clinical diagnosis and appropriate treatment. For example, treatment options for colonic motility disorders are often limited or ineffective, in part due to a poor understanding of the underlying mechanisms of disease.

Currently the only reliable method to investigate the underlying motility patterns associated with colonic disorders is intracolonic high-resolution colonic manometry (HRCM, or alternatively HRM). Previous low resolution colonic manometry catheters were limited by the relative few numbers of recording sites, overall length and flexibility, potentially leading to significant data misinterpretations. The development of high-resolution fiber-optic manometry has led to the much-improved characterization of previously underestimated or unrecognized motor patterns. Recent HRCM studies have revealed a stereotyped markedly increased and sustained occurrence of rhythmic retrograde motor events occurring at ≈2-6 cycles per minute (cpm) most prominently in the rectosigmoid junction after a meal in healthy controls. This retrograde activity in response to ingesting a meal is thought to act as a functional ‘rectosigmoid brake’ which may contribute to reduced rectal filling and therefore normal continence. Abnormalities of Cyclic Motor Patterns (CMPs) are also emerging or hypothesized to contribute to a number of common clinical disorders. However, despite its clinical value, investigation of colonic motility with HRCM is an expensive endoscopic procedure, often done under sedation and invasive, requiring intraluminal insertion of manometry catheters and therefore bowel preparation. Therefore, the uptake of this method in routine clinical practice has been limited to date, thus restricting colonic monitoring and diagnostic potential.

In the past decade, translational studies using high-resolution colonic manometry (HRCM) have found that alterations in colonic CMP activity are implicated in a diverse range of functional bowel disorders. HRCM has become the default tool for researching colonic motility. Modern HRCM have sensors at up to 1 cm resolution, allowing detection of shorter propagating sequences (i.e., CMPs) than previous low-resolution devices. When paired with X-ray imaging, propagating events can be localized to an area along the colon to enable spatio-temporal correlation. However, while HRCM is a valuable research tool, it has not been widely adopted for clinical use due to its limited availability, invasiveness, cost, and complexity of analysis. Other conventional diagnostic tests for colonic functional disorders that are widely available are principally transit studies and do not directly assess motility patterns, limiting the depth of pathophysiological data that can be assessed. The lack of an accessible tool to identify physiological biomarkers has further led to the wide use of symptom-based definitions for functional disorders which impedes proper diagnosis and therapeutic advances.

There is a pressing need for improved and less invasive diagnostic tests that have objective clinical utility, reduce patient harm from negative invasive or unnecessary testing, and directly impact clinical care decisions and treatment. The advent of a less invasive and technically safer diagnostic test for adults and children would broaden availability and access and reduce the high healthcare expenses of current motility testing. An optimal diagnostic solution would be non-invasive, user-friendly, easy to apply and interpret, and provide meaningful diagnostic results that correlate with symptoms and inform appropriate clinical care and treatment.

### BRIEF SUMMARY OF THE INVENTION

The present invention is directed to user-friendly (e.g., patient, clinician) methods and systems for mapping colonic activity in order to provide efficacious and reliable diagnosis and appropriate therapeutic options for patients. Various embodiments of the present invention include non-invasive colonic activity detection systems, such as an electrode array patch and corresponding data acquisition/connector device for mapping colonic activity. Embodiments described herein may be used in the diagnosis and therapy of adults and children by monitoring, analyzing and optimizing measured electrical signals from the non-invasive electrode array patch to provide meaningful results that correlate with symptoms and inform clinical/patient care. Embodiments may also include utilizing real-time patient reported symptoms as a component of the gastrointestinal system for clinical assessment and diagnosis of colonic disorders.

At least some of the embodiments described herein provide a standardized system for quantitative colonic assessment of an individual patient. The system may include continuous or semi-continuous assessment of symptom severity particularly after a meal stimulus for the purposes of diagnostic data collection. Systems described herein use Body Surface Colonic Mapping (BSCM) employing multi-electrode array patches, as described in further detail in U.S. Patent Publication No. 2023-0083795 A1 and U.S. Patent Application No. 63/654,325, which are incorporated herein by reference in its entirety for all purposes, to measure and map colorectal myoelectrical activity. BSCM, or embodiments thereof, may be used in some embodiments of the presentation invention to provide high-quality and high-resolution information non-invasively. Embodiments may also include semi-automated digital and/or analogue tools developed for receiving standardized colorectal symptom profiling.

Embodiments of the present disclosure advantageously provide non-invasive methods and systems for monitoring colonic activity and providing biometric data gained from cyclic motor patterns (CMP) and/or high amplitude propagating contractions (HAPC) data to provide accurate diagnosis and tailored treatments for various colonic diseases. Methods and systems described herein enable detection of one or more biomarkers independently as well as simultaneous detection and differentiation between CMPs and HAPCs to offer significant clinical utility in multiple colonic disorders.

According to one embodiment, a system for mapping colonic and rectal activity of a patient includes an electrode array patch having a plurality of electrodes configured to non-invasively measure electrical signals associated with colonic and rectal activity of the patient over a predetermined time period and a processor configured to receive the measured electrical signals from the electrode array patch over the predetermined time period, determine one or more abnormal cyclic motor patterns (CMPs) based at least in part on the received measured electrical signals, and generate a report comprising at least the determination of the one or more abnormal CMPs. Embodiments of the presently disclosed system gather and provide electrical signals of a patient's colonic and rectal activity without requiring an invasive treatment such as the industry standard colonic manometry procedure.

The system may include various optional embodiments. The processor may be further configured to process the measured electrical signals using a continuous wavelet transform (CWT) analysis to characterize the measured electrical signals within a predetermined frequency bandwidth. The predetermined frequency bandwidth may be between 4 and 10 cycles per minute. The processor may be further configured to compute a body surface colonic mapping (BSCM) motility index (MI) based on the CWT analysis. The BSCM MI may include a CWTmaxval where the CWTmaxval is a mean of a top 10% of wavelet coefficient values at one or more time points during the predetermined time period. The electrode array patch may be disposed on the lower abdomen for measuring electrical signals arising from a rectosigmoid junction, an infraumbilical region, or a left colon. The determination of one or more abnormal CMPs may correlate to a colonic motility disorder. The colonic motility disorder may include fecal incontinence, constipation, irritable bowel syndrome, or low anterior resection syndrome. The predetermined time period may include a meal response duration between 30 minutes and 180 minutes. The processor may be further configured to generate spatio-temporal mapping of the colonic and rectal activity.

According to another embodiment, a method for mapping colonic and rectal activity with an electrode array patch disposed over an abdomen skin surface of a patient includes non-invasively measuring electrical signals associated with colonic activity of the patient with the electrode array patch over a predetermined time period, receiving the measured electrical signals from the electrode array patch over the predetermined time period, determining one or more abnormal cyclic motor patterns (CMPs) and/or abnormal high amplitude propagated contractions (HAPCs) based at least in part on the received measured electrical signals, and generating a report comprising at least the determination of the one or more abnormal CMPs and/or abnormal HAPCs. CMPs and HAPCs can be independently hyperactive or hypoactive, and detecting these abnormalities in concert allows clinicians to differentiate the specific causes of underlying motor dysfunctions and symptoms. The distinction between abnormalities of these patterns independently and vs their coordination is highly valuable for the accurate diagnosis and effective treatment of motility disorders.

The method may include various optional embodiments. The method may further include determining comprises simultaneously detecting CMPs and HAPCs. The method may further include differentiating between CMPs and HAPCs. The report may include a summation image of both the CMPs and HAPCs activity over time. The summation image may include a far field, volume conduction, and/or surface potential diffusion profile. The method may further include determining comprises independently detecting CMPs or HAPCs. The method may further include using a continuous wavelet transform (CWT) analysis to characterize the measured electrical signals within a predetermined frequency bandwidth for each of the CMPs or HAPCs. The predetermined frequency bandwidth may be between 4 and 10 cycles per minute for CMPs. The predetermined frequency bandwidth may be between 0.2 and 13 cycles per minute for HAPCs. The method may further include disposing the electrode array patch on the lower abdomen for measuring electrical signals arising from a rectosigmoid junction, an infraumbilical region, or a left colon. The determination of the one or more abnormal CMPs and/or HAPCs may correlate to a colonic motility disorder. The colonic motility disorder may include fecal incontinence, constipation, irritable bowel syndrome, or low anterior resection syndrome.

According to another embodiment, a system for mapping colonic and rectal activity of a patient includes an electrode array patch having a plurality of electrodes configured to non-invasively measure electrical signals associated with colonic and rectal activity of the patient over a predetermined time period and a processor configured to receive the measured electrical signals from the electrode array patch over the predetermined time period, process the received measured electrical signals using a continuous wavelet transform (CWT) analysis, compute a body surface colonic mapping (BSCM) motility index (MI) based on the CWT analysis to characterize the measured electrical signals as one or more abnormal high amplitude propagated contractions (HAPCs), and generate a report comprising at least the characterization of the one or more abnormal HAPCs. Embodiments of the presently disclosed system gather and provide electrical signals of a patient's colonic and rectal activity without requiring an invasive treatment such as the industry standard colonic manometry procedure.

The system may include various optional embodiments. Using the CWT analysis to characterize the measured electrical signals may be performed within a predetermined frequency bandwidth. The BSCM MI based on the CWT analysis may be a sum of coefficient values of a CWT spectrum generated by the CWT analysis. The predetermined frequency bandwidth may be between 0.2 and 13 cycles per minute. The processor may be further configured to apply a Weiner filter with adaptive variance for removing individual large transient waveforms. The electrode array patch may be disposed on the lower abdomen for measuring electrical signals arising from a rectosigmoid junction, an infraumbilical region, or a left colon. The characterization of the one or more abnormal HAPCs may correlate to a colonic motility disorder. The colonic motility disorder may include fecal incontinence, constipation, irritable bowel syndrome, or low anterior resection syndrome. The predetermined time period may include a meal response duration between 30 minutes and 180 minutes. The processor may be further configured to generate spatio-temporal mapping of the colonic and rectal activity.

According to another embodiment, a system for mapping colonic and rectal activity of a patient includes an electrode array patch having a plurality of electrodes configured to non-invasively measure electrical signals associated with colonic and rectal activity of the patient over a predetermined time period and a processor configured to receive the measured electrical signals from the electrode array patch over the predetermined time period, determine one or more abnormal cyclic motor patterns (CMPs) based at least in part on the received measured electrical signals, determine one or more abnormal high amplitude propagated contractions (HAPCs) based at least in part on the received measured electrical signals, and generate a report comprising at least the determination of the one or more abnormal CMPs or abnormal HAPCs. The report may include a summation image of both the CMPs and HAPCs activity over time. The summation image may include a far field, volume conduction, and/or surface potential diffusion profile.

Other embodiments and variations thereof will become apparent from the following description which is given by way of example only and with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are described by way of example only and with reference to the drawings.

FIG. 1 is an image of a body surface colonic mapping system shown on a patient including a sensing electrode array patch and data acquisition device, in accordance with various embodiments of the present invention.

FIG. 2 is a detailed plan view of the sensing electrode array patch of FIG. 1, in accordance with various embodiments of the present invention.

FIG. 3 is a pictographic representation of steps to set up the gastric activity reader system, a display for logging patient symptoms, and an exemplary symptom report page, in accordance with various embodiments of the present invention.

FIG. 4 illustrates a high-resolution fiber-optic colonic manometer and body surface mapping (BSM) array placement, in accordance with various embodiments of the present invention.

FIG. 5 illustrates HRCM placement, manual markings, and dynamic frequency of CMPs, in accordance with various embodiments of the present invention.

FIG. 6 illustrates motility index temporal correlation grid and time course of the best matching signal processing pipelines, in accordance with various embodiments of the present invention.

FIG. 7 illustrates a summary of CMP meal response match between HRCM and BSCM, in accordance with various embodiments of the present invention.

FIG. 8 illustrates spatial analysis of subject 5 with regional analysis of HRCM MI, in accordance with various embodiments of the present invention.

FIG. 9 illustrates spatial analysis of subject 6 with regional breakdown of HRCM MI, in accordance with various embodiments of the present invention.

FIG. 10 illustrates a box plot summary of the participants' numerically reported outcomes, in accordance with various embodiments of the present invention.

FIG. 11 illustrates an array of BSCM electrical signals correlated with manometry readings showing non-invasive detection of high-amplitude propagating contractions, in accordance with various embodiments of the present invention.

FIG. 12 illustrates BSCM electrical signals including exemplary HAPCs signatures, in accordance with various embodiments of the present invention.

FIG. 13 illustrates a flowchart for assessing BSCM electrical signals for HAPCs signatures, in accordance with various embodiments of the present invention.

FIG. 14 illustrates a flowchart for spatially mapping BSCM electrical signals, in accordance with various embodiments of the present invention.

FIG. 15 illustrates spatial mapping of BSCM electrical signals, in accordance with various embodiments of the present invention.

### DETAILED DESCRIPTION OF THE INVENTION

Abnormal cyclic motor pattern (CMP) activity is implicated in colonic dysfunction, but the only tool to evaluate CMP activity, high-resolution colonic manometry (HRCM), remains expensive, invasive, and not widely accessible. Evaluating colonic activity can be complex relative to other anatomical structures, due to larger anatomical variations, intermittent activity profiles, a more diverse frequency range, and potential for multiple synchronous active regions with independent CMP characteristics. The present disclosure provides signal processing methods and system for body surface colonic mapping (BSCM) for detecting CMPs and/or high amplitude propagating contractions (HAPCs) to accurately diagnose motility disorders and guide therapeutic strategies.

The present invention provides non-invasive assessment of colorectal function using electrophysiological analysis and digital symptom profiling of the gastric and colorectal conduction system to provide actionable biomarkers that stratify patients into therapeutic groups (e.g., such as groups where colorectal dysfunction is present versus absent) to provide a roadmap for personalized (e.g., patient specific) therapy.

Embodiments of the teachings herein may allow for the gathering, combination, and analysis of multiple data sources potentially relevant to understanding colorectal dysfunction. In particular, colorectal activity data is measured (particularly with respect to post meal stimulus), while concurrently gathering temporally synchronized patient symptom information across a test period. Data associated with various embodiments described herein has shown that Body Surface Colonic Mapping (BSCM) biomarkers, to be described in further detail below, are clinically meaningful, because they achieve correlations with symptom severity, which was not achieved by scintigraphy or other tests. BSCM biomarkers and associated gastrointestinal phenotypes as described herein are ideally suited to applications in pediatrics due to their safe and non-invasive nature, and in view of the limited availability and utility of existing diagnostic tests.

Colonic pathophysiology is complex, with diverse putative mechanisms including disorders motility, discoordinated colonic contractions, immune activation, abnormal signaling, autonomic dysfunction, microbiome, psychological (e.g., brain gut influences), visceral hypersensitivity, and impairment of neuromuscular and interstitial cell of Cajal elements, etc. Various embodiments of the present disclosure contribute objective motility diagnostic data, correlating with symptoms, in greater than 60% of patients and, in greater than 90% of those with myenteric/interstitial cell of Cajal (ICC) network pathologies, thereby dramatically improving upon standard of care (e.g. gastric emptying and colonic transit studies; ˜23% detection rate for abnormalities). Such results directly inform clinical management, by stratifying patients into therapeutic groups where gastric and colorectal dysfunction is present versus absent, as a roadmap to personalize therapy.

Various embodiments of the present disclosure describe a non-invasive electrophysiology system designed to independently and/or simultaneously detect and differentiate cyclic motor patterns (CMPs) and high amplitude propagating contractions (HAPCs), offering an advanced diagnostic and therapeutic tool for gastrointestinal motility disorders. The systems of the present invention provide for identification of one or both motor patterns, which are important biomarkers in regulating normal bowel function and are often impaired across various clinical conditions. The presently disclosed system detects the presence, activity/amplitude, rhythm/coordination, and/or spatial locations of these patterns, and presents these data as biomarkers to the clinician as part of a clinical report to help guide appropriate therapeutic strategies for a variety of colonic disorders, including fecal incontinence, constipation, irritable bowel syndrome, and/or low anterior resection syndrome.

CMPs are rhythmic contractile activities most prominently seen in the rectosigmoid junction, sigmoid colon, descending colon, and to a lesser extent in the transverse and ascending colon and rectum. These patterns contribute to the correct coordination of bowel movements. A key component of CMP function is the ‘rectosigmoid brake,’ a retrograde motor pattern that modulates rectal filling and defecation, ensuring bowel control. HAPCs, on the other hand, are large propulsive activities responsible for moving contents through the colon, commonly acting as precursors to defecation.

CMPs and HAPCs can be independently hyperactive or hypoactive, and detecting these abnormalities in concert allows clinicians to differentiate the specific causes of underlying motor dysfunctions and symptoms. In addition, the interplay between CMPs and HAPCs is also important for maintaining normal bowel function, leading to additional clinical relevance. HAPCs events, for example, may be followed by rectosigmoid brake CMP events as a sequence: HAPCs propelling contents toward the rectum, while the CMPs keep them away from the rectum until it is timely to evacuate. The distinction between abnormalities of these patterns independently and vs their coordination is highly valuable for the accurate diagnosis and effective treatment of motility disorders.

In fecal incontinence (FI), for example, hyperactive HAPCs may result in premature defecation, while deficient CMPs, particularly in the rectosigmoid brake, may lead to poor control of rectal filling, with either or both contributing to incontinence. The system as described herein tracks these motor patterns and provides a comprehensive view of the bowel's motor functions, enabling clinicians to pinpoint whether incontinence is primarily due to abnormal HAPCs or a failure of the CMPs, especially the rectosigmoid brake, for more targeted interventions. For example, sacral neuromodulation (SNS) is a commonly effective and practiced intervention for incontinence. Methods of sacral nerve stimulation therapy may include at least some embodiments as described in U.S. Pat. No. 11,712,566 which is hereby incorporated by reference in its entirety for all purposes. Upregulating CMP activity may be used to restore rectosigmoid brake function. The present system provides objective biomarkers for CMPs and HAPCs to assess therapeutic success and thereby guide patient selection. Sometimes, an excessive result from SNS in a patient with a normal rectosigmoid brake can even lead to rebound constipation. Monitoring such results through the presently disclosed diagnostic system can help to personalize and tailor SNS protocols and therapies to individual results. In other cases, the SNS stimulus amplitude may need to be increased to heighten CMP activity further for improved efficacy.

In constipation, hyperactive or irregular CMPs may over-regulate or impair bowel function, inhibiting effective propulsion and leading to stasis, whereas diminished HAPCs indicate impaired propulsive capability. It is currently impossible for clinicians to determine which of these mechanisms is responsible for an individual patient's constipation. By distinguishing between these dysfunctions, the presently disclosed system may guide treatment choices, such as whether prokinetics or laxatives or antispasmodics would be more beneficial. Furthermore, the presently disclosed system's ability to demonstrate severely impaired motility (e.g., significantly reduced HAPCs and/or disrupted CMPs refractory to medical therapies, as monitored by the novel biomarkers) assists in making decisions regarding surgical interventions, such as colonic resection, for patients with refractory constipation.

In irritable bowel syndrome (IBS), which includes subtypes such as IBS-C (constipation predominant), IBS-D (diarrhea predominant), and mixed forms, the presently disclosed system aids in determining whether symptoms arise from abnormal motor patterns or a sensory disorder. For example, spasmodic CMP activity may underlie basal motor dysfunction, while abnormal HAPCs could trigger episodic symptoms like diarrhea or urgency. In cases where both CMPs and HAPCs are normal, the focus may shift to therapies addressing sensory dysfunction, such as neuromodulators, dietary adjustments, or psychological interventions. Additionally, the ability to differentiate motor involvement in constipation or diarrhea subtypes allows for more precise and individualized therapy recommendations.

For low anterior resection syndrome (LARS), large anatomical bowel regions responsible for CMPs may have been resected (such as for rectal cancer therapy), meaning the CMPs are reduced. Alternatively, symptoms could be due to issues such as sensory disorders, capacity disorders or sphincter dysfunction. The present disclosed system's ability to detect abnormal CMPs following surgical resection provides valuable data for guiding post-operative care. LARS is the major determinant of quality of life following colorectal cancer and is particularly prevalent in patients requiring radiotherapy. Disruption of normal motility patterns after surgery/radiotherapy often leads to erratic bowel movements, and the system offers clinicians detailed insights into whether these disturbances are due to basal motor dysfunction or abnormal propulsive events, or other causes. This information is key for developing tailored treatment plans aimed at restoring more normal motility in post-resection patients, for example, dietary therapies, vs SNS, vs laxatives or irrigation therapy.

Overall, the presently disclosed system's capacity to synergistically detect and differentiate between CMPs and HAPCs offers significant clinical utility in multiple disorders. It provides clinicians with the data necessary to accurately diagnose motility disorders and tailor therapeutic strategies, accordingly, potentially improving patient outcomes across a range of gastrointestinal conditions.

Various embodiments of the present disclosure include a medical apparatus for monitoring electrical activity including a sensing device such as an electrode patch or a plurality of patches having one or more electrodes and a connector device (or devices) which may be an electronic device such as a data acquisition device that is in electronic communication with such patch. Advantageously, various embodiments of the present disclosure provide an electrode patch connection system for a non-invasive medical apparatus that may be worn by a subject to monitor the physiological condition in a comfortable and reliable manner, while the subject is engaged in normal daily activities, and/or in a clinical test setting.

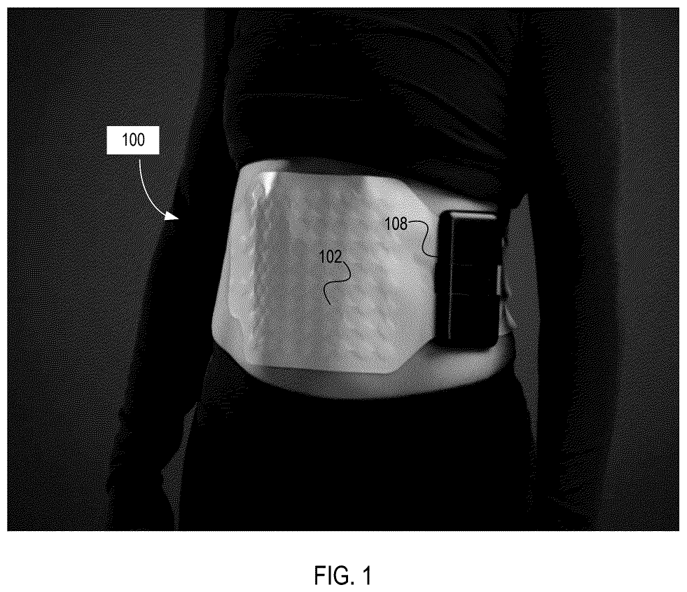

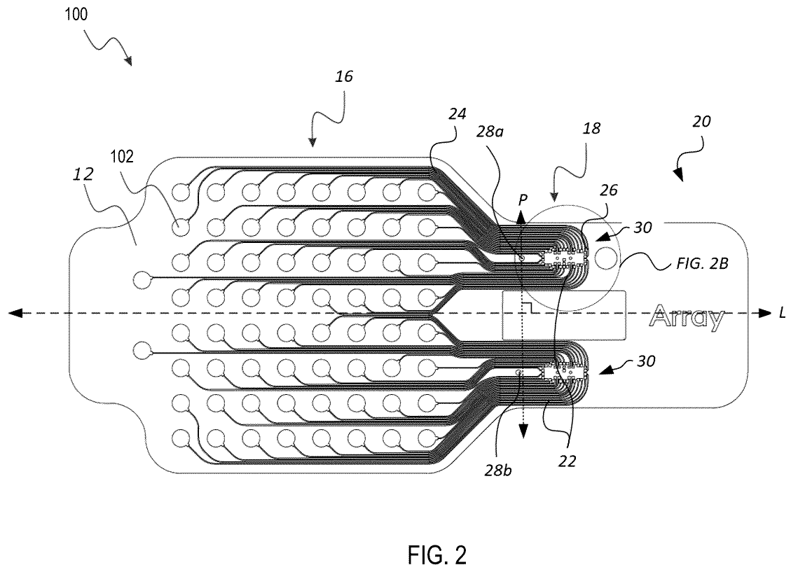

FIGS. 1 and 2 illustrate an exemplary electrode patch 100 for monitoring physiological functions on a subject. According to various embodiments, the subject is a human in some implementations but optionally the subject may be a non-human animal. The electrode patch 100 is configured in some embodiments to be used as part of a system for monitoring electrical activity of a subject. In some embodiments, the electrode patch 100 may be configured to monitor electrical/physiological activity of colonic regions including, but not limited to, the ascending, transverse, descending or sigmoid regions. In exemplary embodiments, the electrode path 100 is positioned on the lower abdomen primarily to capture electrical activity arising from the rectosigmoid junction and the left colon.

The electrode patch 100 is a sensing device and may include a plurality of spatially arranged surface electrophysiological sensors in the form of electrodes 102 for contacting an outer surface of the skin of the subject to sense and measure electrical potentials at multiple electrodes. Embodiments of the electrode patch 100 are not to be limited by the exemplary embodiment shown in FIG. 1.

As shown in the exemplary embodiment of FIG. 2, there are total of 66 electrodes out of which 64 electrodes are arranged in an array of 8 rows and 8 columns and the remaining two electrodes are the ground and reference electrodes. In use, electrical potentials may be measured as the difference between each of the 64 electrodes and the reference electrode. The ground electrode may be the “driven right leg” or “bias” electrode. The purpose of the ground electrode in some embodiments is to keep voltage level of the subject's body within an acceptable range and to minimize any common-mode in the subject's body (e.g., 50/60 Hz power-line noise). The driven right leg may act as a source or sink. However, the electrode patch 100 may comprise more than 66 electrodes or less than 66 electrodes. The ground and reference electrodes may be different than what is shown in FIG. 2. In an embodiment, the patch may comprise less than, greater than, or equal to 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275 or 300 electrodes any value or range of values therebetween in 1 increments (e.g., 33, 94, 44 to 192, etc.).

In some embodiments, the electrode patch 100 is configured to be removably attached to the outer surface of the skin of the subject, such as at or near an abdominal region (as shown in FIG. 1), so that the electrodes 102 contact the outer surface of the skin of the subject at or near the abdominal region to sense and measure electrical signals from the GI tract of the subject. If the electrode patch 100 is for sensing and measuring electrical signals from other regions, then the electrode patch may be configured to be removably attached to the outer surface of the skin of the subject at or near at suitable regions, so that the electrodes 102, contact the outer surface of the skin of the subject at or near such region to sense and measure electrical signals from the that region of subject's body.

In various embodiments, the electrode patch 100 and data acquisition system may be as described in International Patent Application Publication No. WO 2021/130683 which is hereby incorporated by reference in its entirety for all purposes. For example, the electrical traces 106 may connect each electrode 102 and/or to a respective contact pad 104, for operatively coupling with a data acquisition device 108 (interchangeably referred to herein as a connector device, a connection device, etc.). For example, the data acquisition device 108 may be coupled to the electrode patch 100 and wirelessly coupled to a processor. The data acquisition device 108 may be configured for transmission of the measured electrical signals to the processor. Furthermore, the system may include a patient mobile device (e.g., a smart phone, tablet, or the like) for patient symptom information input and the patient mobile device may be in wireless communication (e.g., Bluetooth or the like) with the processor for transmission of patient symptom information. The system may comprise a docking device having a compartment that is configured to receive the data acquisition device of the sensor array. The docking device may be a wireless charging device for facilitating wireless charging of the data acquisition device when docked. The electrode patch and data acquisition system enable body surface colonic mapping (BSCM) information to be received in an autonomous or semi-autonomous manner. Additionally, the system may include a display for displaying a generated report as described in further detail herein. The display may be part of the patient mobile device or of a separate device used by the health care professional.

In some embodiments, an electrode patch according to embodiments described herein may be used to measure colonic activity in response to a meal stimulus. In further embodiments, testing is implemented through a standardized system to output high quality data and data for comparison purposes. A test protocol may include that the participant fast for at least 6 hours and avoid medications modifying gastric and colorectal function as well as caffeine and nicotine on the day of testing. Embodiments may include fasting for at least 2, 3, 4, 5, 6, 7, 8, 9, 10 hours or more or any value or range of values therebetween in 15-minute increments. Tests may be, in some embodiments, conducted in the morning. The fasting may be linked to the onset of testing (e.g., fasting for at least 2 hours would correspond to starting the testing 120 minutes after food was last consumed).

In various embodiments, an electrode patch according to embodiments described herein may be used to measure colonic activity in over an extended time period. For example, the patient may be mobile and at home, with the presently disclosed system recording while the patient undertakes routine daily activities including eating and digesting one or more meals.

FIG. 2 is a top view of the flexible electrode patch. In particular, FIG. 2 is a top view of the flexible electrode patch 100 of FIG. 1 and the description of FIG. 1 is relevant to the present description of FIG. 2 unless otherwise noted herein. As shown in FIG. 2, the plurality of electrically conductive contact pads 26 may all be present in a connector region 18 of the flexible substrate 12. The connector region 18 may be configured for the releasable attachment of a data acquisition device (not shown) which electrically connects to the plurality of electrically conductive contact pads 26. The connector region 18 may include the one or more alignment holes 28 for this purpose. The data acquisition device may attach by clamping over the plurality of electrically conductive contact pads 26 and the one or more alignment holes 28. The plurality of electrically conductive contact pads 26 may be arranged in one or more clusters 30 to improve ease of aligning the data acquisition device.

As illustrated in FIG. 2, the one or more alignment holes 28 are offset for emphasizing the correct orientation of a data acquisition device relative to the flexible electrode patch 10. As shown, alignment hole 28a and alignment hole 28b are offset from each other along an axis P that is perpendicular to a longitudinal axis L of the flexible electrode patch 10. Accordingly, a data acquisition device having corresponding projections for inserting into alignment hole 28a and alignment hole 28b may be correctly positioned only when the data acquisition device is in a correct orientation relative to the flexible electrode patch 100 such that the projections align with the one or more alignment holes 28.

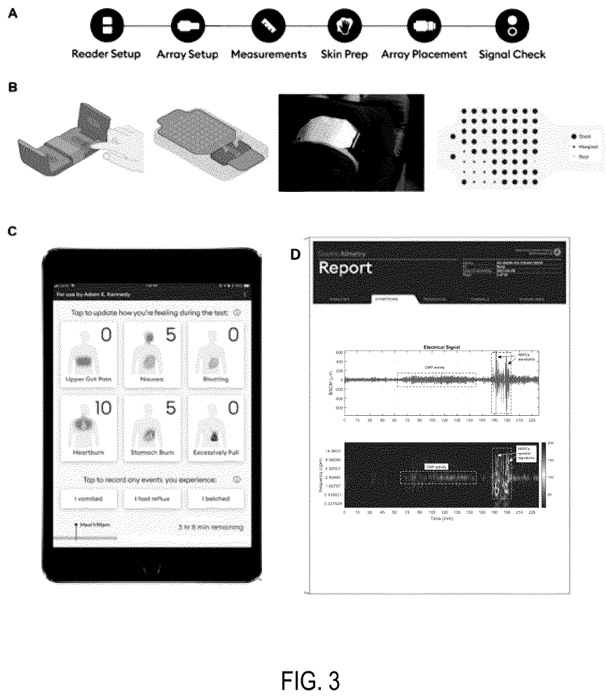

FIG. 3 is a pictographic representation of steps to set up the colonic activity reader system. Part A details basic steps to prepare array on a patient. Part B illustrates coupling of an array with a reader/data acquisition device, and electrode signal check. Part C represents a patient mobile input device (e.g., a tablet computer or the like) for a mobile application used by a patient, for example, to log real time (and concurrent) symptom data. For example, a patient may input incidents and severity of symptoms including one or more of lower gut pain, urgency, soiling, bloating, spasm, flatus, etc. The patient may input symptom data at predetermined time intervals (e.g., every 5 minutes, every 10 minutes, every 15 minutes, etc.) and the patient may input symptom data as symptoms occur. Part D illustrates an example report including a spectral map showing simultaneous detection of both CMPs and HAPC using the methodologies described, within a single image. The exemplary report includes a summation image of both the CMPs and HAPCs activity over time. The summation image may include a far field, volume conduction, and/or surface potential diffusion profile. For example, the surface potential diffusion profile may describe how waves spread out differentially based on wavelength and frequency. Diffusion describes the more general notion of source (e.g., colonic) blurring when measuring on the body surface.

Sensor array placement may be preceded by shaving, followed by skin preparation with an exfoliant conductive gel such as NuPrep® (Weaver & Co, CO, USA) to minimize impedance. According to some embodiments, a mobile application for use with electrode array patch and the reader/connection device may be provided for performing an impedance threshold check prior to allowing recording (see FIG. 3, part A). Fasted recordings may be performed for 30 minutes, for example, followed by standardized meal consumed over a predetermined time period (e.g., 10 minutes). In some embodiments, a predetermined test period up to a 4-hr postprandial recording is performed. For example, a 4-hr postprandial recording period may capture a full gastric and colorectal activity cycle including meal responses occurring 2-4 hrs after a meal. In other embodiments, a predetermined test period may be 30 to 60 minutes, and preferably around 45 minutes. Accordingly, in an embodiment, fasted recordings are performed for less than, greater than, or equal to 10, 15, 20, 25, 30, 35, 40, 45, 50, 55, 60, 65, 70, 75 or more minutes or any value or range of values therebetween in 1-minute increments, contiguous. In some embodiments there is postprandial recording period of less than, greater than, or equal to 0.2, 0.25, 0.5, 0.75, 1, 1.5, 2, 2.5, 3, 3.5, 4, 4.5, 5, 5.5, 6, 6.5 or 7 hours or more or any value or range of values therebetween in 0.5-minute increments. According to an exemplary embodiment, a predetermined time period may include one or more of a 30 minute fasting period, a 10 minute meal window, and a 4 hour post-prandial observation.

In various embodiments, a standard test meal may comprise an off-the-shelf nutrient drink (e.g., Ensure 232 kcal, 250 mL; Abbott Nutrition, IL, USA) and oatmeal energy bar (e.g., a Clif Bar with 250 kcal, 5 g fat, 45 g carbohydrate, 10 g protein, 7 g fiber; Clif Bar & Company, CA, USA). In exemplary embodiments, the calorie consumption of the standard meal is less than, greater than, or equal to 150, 200, 250, 300, 350, 400 or 450 kcal or any value or range of values therebetween in 10 kcal increments. In an embodiment, a standardized meal is consumed within less than, greater than, or equal to 30, 25, 20, 15, 10, 9, 8, 7, 6 or 5 minutes or any value or range of values therebetween in 1-minute increments continuous from beginning to end. In some embodiments, the fat, carbohydrate, protein and/or fiber may have a nutritional value less than, greater than, or equal to 30, 35, 40, 45, 50, 55, 60, 65, 70, 75, 80, 85, 90, 95, 100, 125, 150, 175 or 200% or more or any value or range of values therebetween in 1% increments. The fat, carbohydrate, protein and/or fiber may have varied amounts thereof.

According to various embodiments, meals with similar nutritional composition may be substituted per availability or for patients with specific dietary needs, such as those with diabetes or gluten intolerance. For example, various embodiments described herein may be used in combination with testing for monitoring and managing blood sugars in diabetics during testing as hyperglycemia may induce colon motility abnormalities. In various embodiments, the standardized meal is designed to stimulate colonic symptoms in patients with diverse colonic disorders, including irritable bowel syndrome. In some embodiments, the percent of the standardized meal that is consumed is less than, greater than, or equal to 25, 30, 35, 40, 45, 50, 55, 60, 65, 70, 75, 80, 85, 90, 95, 100, 125, 150, 175 or 200% or more or any value or range of values therebetween in 1% increments.

In various embodiments, nothing is consumed for greater than, or equal to 0.5, 1, 1.5, 2, 2.5, 3, 3.5, 4, 4.5, 5, 5.5 or 6 hours or more or any value or range of values therebetween in 3-minute increments before and/or after the aforementioned timeframe. In an embodiment, only de minimus foods are consumed (e.g., a mint for example) within those times, while in other embodiments, nothing is consumed.

Various embodiments include minimizing movement, talking, sleeping and avoiding touching the electrode array patch to reduce artifact contamination, other than overlying clothes or blankets, etc. In some embodiments, patients are positioned in a comfortable chair that is reclined at 30, 35, 40, 45, 50, 55, 60, 65, 70 or 75 degrees or any value or range of values therebetween in 1-degree increments, and in some embodiments, with their legs elevated, to reduce and/or avoid abdominal wall contractions. In some embodiments the selected chair may be locked in a set reclined position, or at least prevented from moving more than a certain range (e.g., within 1, 2, 3, 4, 5, 6, 7, 8, 9, 10, 11, 12, 13, 14, 15, 16, 17, 18, 19, 20, 21, 22, 23, 24 or 25 degrees or any value or range of values therebetween in 1 degree increments), so as to reduce restless abdominal tensing which may contaminate data with electromyographic noise. During the test, patients may move for comfort adjustments or bathroom breaks with, in at least some embodiments, an on-board accelerometer data being tracked to identify periods of motion.

Temporal associations between physiological events and symptoms may be used to inform mechanistic interpretations. Accordingly, a patient symptom-logging application on a mobile smart phone or tablet or the like (such as shown in FIG. 3, part C) is provided in at least some embodiments to differentiate symptoms with severity lying on a continuum or specific events. In other embodiments, patient symptom information may be collected manually and later entered into the system for quantification and analysis.

For example, symptoms including one or more of lower gut pain, urgency, soiling, bloating, spasm, flatus, etc., may be assessed on a continuum and/or discrete events of the foregoing symptoms may be time stamped.

Continuous symptoms are assessed during the test at suitably granular intervals. For example less than, greater than, or equal to 2, 3, 4, 5, 6, 7, 8, 9, 10, 11, 12, 13, 14, 15, 16, 17, 18, 19, 20, 21, 22, 23, 24, 25, 26, 27, 28, 29 or 30 or any value or range of values therebetween in 1 increment minute intervals may be used in some implementations. In an embodiment, 10, 15, 20, 25, 30, 35, 40, 45, 50, 55, 60, 70, 80, 90, 100, 125, 150, 175 or 200 or more or any value or range of values therebetween in one increment assessments are made during the test. In an embodiment, the assessments are spaced apart by any one or more the time intervals.

Symptom information may be entered via a pictographic interface (such as on a GUI of a computer or smart phone or smart device, etc.) that aids accurate standardized reporting, for example using a 0-10 visual analog scale (where 0 indicates ‘no symptom’ and 10 indicates the ‘most severe extent’ of a symptom). A speech to text system may be used where the patient describes the experience. Any responses may be time stamped. The patient may be prompted by the computer or smart device, such as audibly or in a tactile and/or visual manner, etc.

Various embodiments of the present disclosure include processing measured electrical signals received at a processor from the electrode patch 100. According to various embodiments, the length of a CMP (units of cm) was defined as the distance between the first and last manometer sensors along the same marked sequence, e.g., 5 consecutive sensors involved making up a CMP=4 cm in length. The instantaneous rate of CMPs may be determined from the number of propagating events occurring within a sliding 2 min window. To prevent double counting, a single propagating event may be time-stamped at its mid-point. HRCM frequency may be analysed according to various methods. Intrinsic frequency may be defined as the rate of CMP activity detected on each individual manometer sensor, independent of other sensor's data, thus representing the electrophysiological rate of region-specific CMP activity. Sequential frequency may be defined using the time interval of 2 successive events marked array-wide on any sensor, thus representing the rate of multifocal CMP activities along the entire colon superposing at the body surface. When only one region is active, the sequential frequency equals the intrinsic frequency. According to the present disclosure, HRCM frequency refers to the intrinsic frequency unless stated otherwise.

An optimized BSCM signal processing method may include using a Continuous Wavelet Transform (CWT) analysis to characterize the electrical activities occurring within a predetermined frequency bandwidth. Initially, three frequency bandwidth filters were used to cover the colonic frequency ranges previously stated in literature and observed in the HRCM results in this study; low (0.6-6 cpm), high (5-12 cpm) and wide (0.12-12 cpm). A BSCM motility index (MI) was derived from the CWT analysis in a 3-step process. First, for each channel the CWT spectrogram was computed. The magnitude of CWT coefficients may represent the amplitude of the signal at each frequency (scale) and time point. Second, the array-wide spectrogram was computed by averaging CWT spectrograms from all individual-channels.

A ‘CWTmaxval’ method may include computing the BSCM MI as the mean of the top 10% wavelet coefficient values at each time point. Because the preprocessing stage accurately rejects artifacts and noise components, CWTmaxval is a robust metric quantifying the colonic motility over time, which may exhibit a variable dominant frequency.

### Methods and Testing

This study aimed to validate body surface colonic mapping (BSCM) through direct correlation with HRCM. Synchronous meal-test recordings were performed in asymptomatic participants with intact colons. A signal processing method for BSCM was developed to detect CMPs. Quantitative temporal analysis was performed comparing the meal responses and motility indices (MI). Spatial heat maps were also compared. Post-study questionnaires evaluated participants' preference and comfort/distress experienced from either test. 11 participants were recruited and 7 had successful synchronous recordings (5 females/2 males; median age: 50 years [range 38-63]). The best-correlating MI temporal analyses achieved a high degree of agreement (median Pearson correlation coefficient (Rp) value: 0.69; range 0.47-0.77). HRCM and BSCM meal response start and end times (Rp=0.998 and 0.83; both p<0.05) and durations (Rp=0.85; p=0.03) were similar. Heat maps demonstrated good spatial agreement. BSCM is the first non-invasive method to be validated by demonstrating a direct spatio-temporal correlation to manometry in evaluating colonic motility.

### High-Resolution Colonic Manometry

A fiber-optic manometry catheter with 72 sensors spaced at 1 cm intervals was used. All participants agreed to have placement in the same procedure as the elective colonoscopy. Following the clinical colonoscopy, a re-entry was made with an endoscopic grasper holding a nylon loop which had been placed at the proximal tip of the manometer. The catheter was placed in the maximum possible extent in the right colon. At extent, the nylon loop was endo-clipped to the colonic mucosa to keep the catheter in place and the colonoscope was gently removed. The manometer was fixed in position with an adhesive dressing to the right buttock to prevent unintended withdrawal during the recording period. A spectral interrogator acquisition unit was connected to the catheter to record the data (FBG-scan 804; FOS&S, Geel, Belgium).

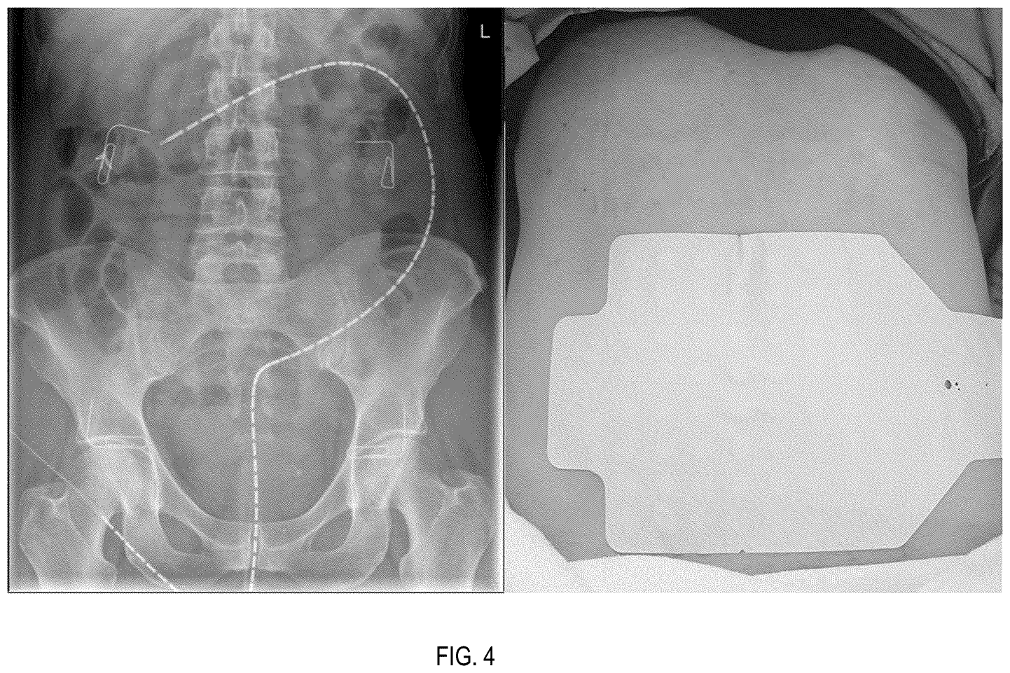

FIG. 4 illustrates a high resolution fiber-optic colonic manometer and body surface mapping (BSM) array placement. On the left, 72 sensor manometer is shown with 1 cm sensor spacing was clipped with multiple endoclips to the mucosa of the colon. The paperclips demarcate the area covered by the 8×8 electrode grid of the array. On the right, a BSCM array is affixed to abdominal skin. Ground and reference electrodes are located on the small extension flap of the array on the right (anatomical) side of the participant.

### Body Surface Colonic Mapping

The BSCM electrical recordings were acquired using the Alimetry body surface mapping hardware (Alimetry Ltd, Auckland, New Zealand) with an 8×8 stretchable electronics adhesive array with pre-gelled Ag/AgCl electrodes at 2 cm spacing; area 225 cm². Prior to array placement, abdominal hair was clipped, and the skin was prepared with an exfoliant (NuPrep Weaver, Aurora, CO) to facilitate low impedance electrical contact. The array was positioned on the lower abdomen primarily to capture electrical activity arising from the rectosigmoid junction and the left colon.

### Experimental Protocol

On the morning of the experiment, informed consent was obtained. All participants completed the standard regimen of the bowel purgative agent Glycoprep-C® (Fresenius Kabi, Australia). The choice and dose of procedural medications (anesthetic and analgesia) were decided by the endoscopist/anesthetist. Smooth muscle relaxants, such as hyoscine, were not administered. Following placement, the manometer was connected to the acquisition unit, and the BSCM array was placed on the abdomen and connected to the Alimetry Reader. Participants underwent a plain abdominal X-ray with the corners of the array marked with radio-opaque clips to spatially register the position of the manometer in relation to the body surface array. Concurrent data acquisition was performed for three to four hours (one hour pre-meal period, two to three hours post-meal). During the recording, participants were asked to lie in a reclining chair/bed positioned 20-30 degrees from horizontal. The standardized meal included a 232-kcal nutrient drink (230 ml Ensure; Abbott Nutrition, IL, USA) and an oatmeal energy bar (250-kcal with 5 g fat, 45 g carbohydrate, 10 g protein, 7 g fiber; Clif Bar & Company, CA, USA). Participants were given 10 min to complete the meal. At the end of the study participants were asked to fill out an electronic questionnaire pertaining to the comparative experience of HRCM and BSCM. Likert scales from 1 to 10 were used for perceptions of discomfort/pain and usability.

### Data Analysis

The protocol included a 10 min settling period, which was not analyzed. The earlier end of data acquisition of either the HRCM or BSCM device was deemed the end of time of the experiment.

### HRCM Analysis

Primary analysis was performed using PlotHRM (Flinders University, Adelaide, Australia). Markers were placed manually on the consecutive peaks of propagating or simultaneous events. Criteria for motor events were pressurizations of 5 mmHg or greater across four or more consecutive channels (i.e., 4 cm or longer). All pressure events in multiple (2 or greater) within 1 min of each other were marked and included in the analysis. Custom MATLAB R2022b (MathWorks, Natick, Massachusetts, USA) software was used for further analysis. Amplitude (in units of mmHg) was defined, following previous studies, as the average of the peak pressures noted in every channel involved in a propagating pressure wave. Length of a CMP (units of cm) was defined as the distance between the first and last manometer sensors along the same marked sequence, e.g., 5 consecutive sensors involved making up a CMP=4 cm in length. The instantaneous rate of CMPs was determined from the number of propagating events occurring within a sliding 2 min window. To prevent double counting, a single propagating event was time-stamped at its mid-point. HRCM frequency was analyzed using two separate methods. Intrinsic frequency was defined as the rate of CMP activity detected on each individual manometer sensor, independent of other sensor's data, thus representing the electrophysiological rate of region-specific CMP activity. Sequential frequency was defined using the time interval of 2 successive events marked array-wide on any sensor, thus representing the rate of multifocal CMP activities along the entire colon superposing at the body surface. When only one region is active, the sequential frequency equals the intrinsic frequency. HRCM frequency refers to the intrinsic frequency unless stated otherwise.

### BSCM Analysis: Preprocessing Methods

An optimized BSCM signal processing method was developed on the foundations of a previous proof of concept study that used Continuous Wavelet Transform (CWT) analysis to characterize the electrical activities occurring within a set frequency bandwidth. Guided by HRCM frequency analysis and other literature, it became prudent to further develop the signal processing pipeline to be capable of monitoring a wide and dynamic frequency range at the body surface. Initially, three frequency bandwidth filters were used to cover the colonic frequency ranges previously stated in literature and observed in the HRCM results in this study; low (0.6-6 cpm), high (5-12 cpm) and wide (0.12-12 cpm). Subsequently, frequency ranges were fine-tuned on an empirical basis. For each of 10 total frequency bands tested, it was explored whether application of common mode re-referencing (CMR) and linear minimum mean square estimation (LMMSE) artifact reduction improved the correlation with HRCM MI. In total, it was explored a parameter space consisting of 40 total preprocessing combinations.

Raw BSCM recording were processed as follows:
1. Remove baseline wander using a moving median filter with a 30 s window.
2. If selected, reduce motion artifacts using LMMSE filter with 30 s averaging window and adaptive noise threshold window of 300 s.
3. Attenuate noise and interference while isolating various putative colonic components using a Butterworth filter (2nd order; zero phase). Passbands (in units of cpm): 0.12-12, 0.6-6, 0.6-12, 2-8, 3-10, 4-10, 4-12, 5-8, 5-12, and 8-12 were tested.
4. If selected, apply common mode re-referencing (CMR) following the PREP pipeline. CMR was applied using only channels with good signal quality, based on criteria of low electrical impedance (<500 kΩ); low noise but not “dead channel” amplitude (1 to 4000 uV); sufficient spectral correlation to other channels (correlation>0.3). Only ‘good’ channels meeting all of the above criteria were included for the remaining analyses; ‘bad’ channels were excluded.
5. An additional stage of large transient reduction was applied based on soft-thresholding. The soft-thresholding coefficient was determined as:

$$λ = 1 - {( \frac{s ( t )}{n ( t )} )}^{2} ; 0 \leq λ \leq 1 \vert \qquad ( 1 )$$

where s(t) is the magnitude of the Hilbert Transform of the signal resulting from steps 1-4 above, smoothed over a 300 s window. It's ratio relative to n(t)=median (s(t))+k*MAD(s(t)) sets the threshold level, where MAD is the median of the absolute deviation, an estimate of the signal variance. The method may include setting k=5, as well as a maximum value for n(t)=500 uV for compatibility with physiologically plausible maximum signal strength. Note that colonic signal amplitudes are expected to be in the range of 50-200 uV, such that non-artifact corrupted segments of the signal amplitude are only modestly attenuated by ˜1-16%, while larger transients are strongly reduced.

### Motility Indices: HRCM MI

The study focused on aspects of CMPs that should be detectable from the body surface including temporal activation and localization of the regions of CMP activity. Specific characteristics of any individual motility events, such as direction and velocity of a CMP, were judged less likely to be relatable to BSCM electrophysiological signals owing to complex orientation and geometry of the colon and the spatial volume conductor effect. Thus, the HRCM motility index (MI) was defined as representing the summative CMP activity as a product of three metrics: number of CMPs per unit time (N_CMP); mean amplitude (A) and distance of propagation (L):

$$HRCM M 1 = {N}_{CMP} \times L ( cm ) \times A ( mmHg ) \qquad ( 2 )$$

### Motility Indices: BSCM MI

BSCM MI was derived from Continuous Wavelet Transform (CWT) analysis in a 3-step process. First, for each channel the CWT spectrogram was computed as previously described. Note the magnitude of CWT coefficients represent the amplitude of the signal at each frequency (scale) and time point. Second, the array-wide spectrogram was computed by averaging CWT spectrograms from all individual-channels. Finally, BSCM MI was computed as the mean of the top 10% wavelet coefficient values at each time point, referred to herein as the ‘CWTmaxval’ method. Because the preprocessing stage accurately rejects artifacts and noise components, CWTmaxval is a robust metric quantifying the colonic motility over time, which may exhibit a variable dominant frequency.

### Comparative Analysis Outcomes

Temporal (quantitative) analysis. Meal response: Meal response start, end and duration times were assessed independently by 2 researchers (SHBS and JE) and any disagreements were resolved by a third reviewer (CW). Meal responses start time was the earliest time at which MI rose above the baseline level for longer than 10 min. The point at which the MI returned to baseline levels, sustained for the ensuing 10 min or long, was the end time of the meal response. Motility index correlations: Correlation analyses evaluated which BSCM MI result for each of 40 preprocessing parameter sets (10 filter bandwidths×2 artifact reduction settings (on/off)×2 CMR settings (on/off)) best represented the CMP activity as apprised by manometry. HRCM MI and BSCM MI were smoothed over a 5 min window, chosen to reduce noise while preserving temporal dynamics characteristic of episodic CMPs observed in this study cohort.

Spatial correlations. The abdominal X-ray images were used to register the approximate location and orientation of the manometer and the body surface array. To spatially compare the dominant region(s) of CMP activity, the experiment time course was divided into manually identified epochs that were considered to be major phases of the meal response; pre-meal, meal response, post meal response (quiescence) and secondary active segments (if any) as identified from HRCM analysis. Spatiotemporal activity in each of these time periods were compared using HRCM activity maps to BSCM heatmaps, visualizing the most active zones detected in each, respectively. Qualitative analyses assessed the synchronicity and general location of dynamic spatial ‘hot spots’ during the meal response epochs. Temporal epochs for each study participant were manually identified through multifactorial HRCM analyses. Each study contained at least a pre-meal and meal-response phase. For some subjects, the post-meal response was multi-phasic: following the primary meal response, an epoch of quiescence was followed by a secondary period of activity.

HRCM spatiotemporal activity maps were generated in each temporal epoch using a two-step process as follows:
1. The cumulative mean activity for each sensor was computed as

$$S = \frac{1}{T} \cdot {\sum }_{k} {P}_{k}$$

where Pk denotes pressure amplitude of the k-th marked event within the time window of duration T.
2. Bivariate kernel smoothing was applied with a bandwidth equal to ˜1.5% of the mean scale of the X-ray image (in pixels). This last step was done primarily to match the approximate diameter of the colon in the X-ray image.

BSCM heatmaps were rendered for each temporal epoch as follows:
1. For each good channel, the MI was scaled to have unit area under the curve: MI_{BSCM}(t)/ΣₜMI_{BSCM}(t). Normalization helps compensate for variable source to sensor distance across the array, emphasizing overall changes in activity.
2. The mean MI for within a defined temporal epoch was computed for each electrode.
3. To fill gaps in the map where bad electrodes existed, inverse cubic weighting interpolation (distance scale=2 pixels) temporal weighting was applied with a radius of 2×electrode distance (=4 cm).
4. To reduce the effect of any remaining outliers in the heat map, a thin plate spline was applied (smoothing parameter=0.5).
5. The 8×8 grid of values was up-sampled by a factor of 10× for a clearer visualization.
6. Global color scale limits were set using the 25-99 percentile of values in the heat map. These values were empirically determined to be a good compromise between sufficiently rendering detail in a single map versus highlighting large contrasts in activity that may exist across epochs (e.g., quiescent vs active).

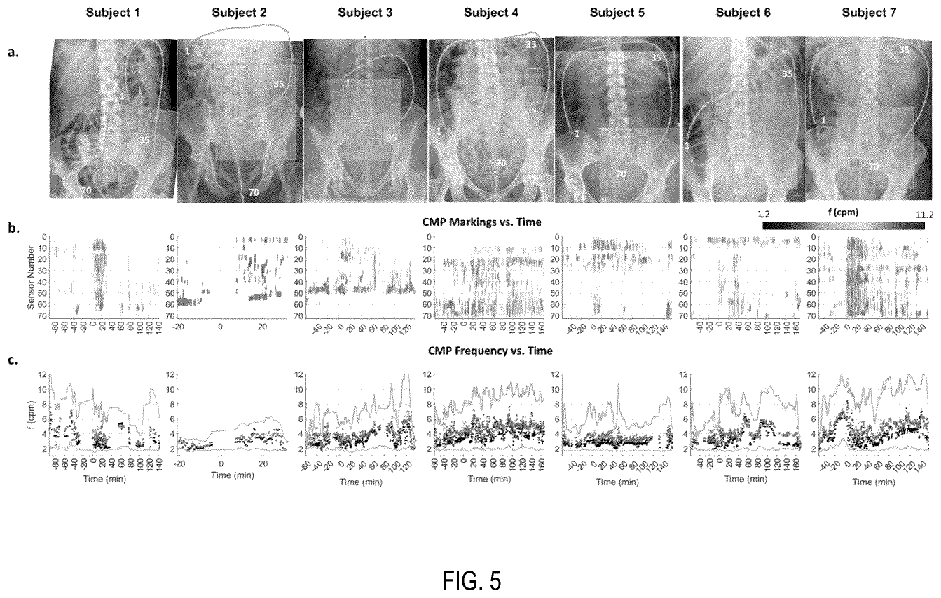

FIG. 5 illustrates HRCM placement, manual markings, and dynamic frequency of (a) X-ray images of each participant showing the extent the manometer (orange line) reached. Sensor numbers 1, 35, and 70 are annotated. (b) Manually marked CMP events graphically stacked with the most proximal sensor located at the top of the vertical axis (sensor number 1). The time axis is displayed with the meal start aligned at t=0. CMP events are color coded according to intrinsic frequency (linear scale of 1.2 cpm [deep blue] to 11.2 cpm [red]) to highlight the dynamic frequency changes. Densely marked (pacemaker) regions of CMP activity can also be appreciated and anatomically localized which can then be used to develop a spatial ‘heatmap’ along the length of the manometer. Multi-focal activity can be observed as clusters of CMP marks that are vertically discrete with minimal activity occurring in the sensors separating them. (c) Using the data from panel b, frequency time course maps were developed. Orange dots indicate the mean frequency, black indicates the median frequency, and the gray lines indicate the 10-90 percentile range.

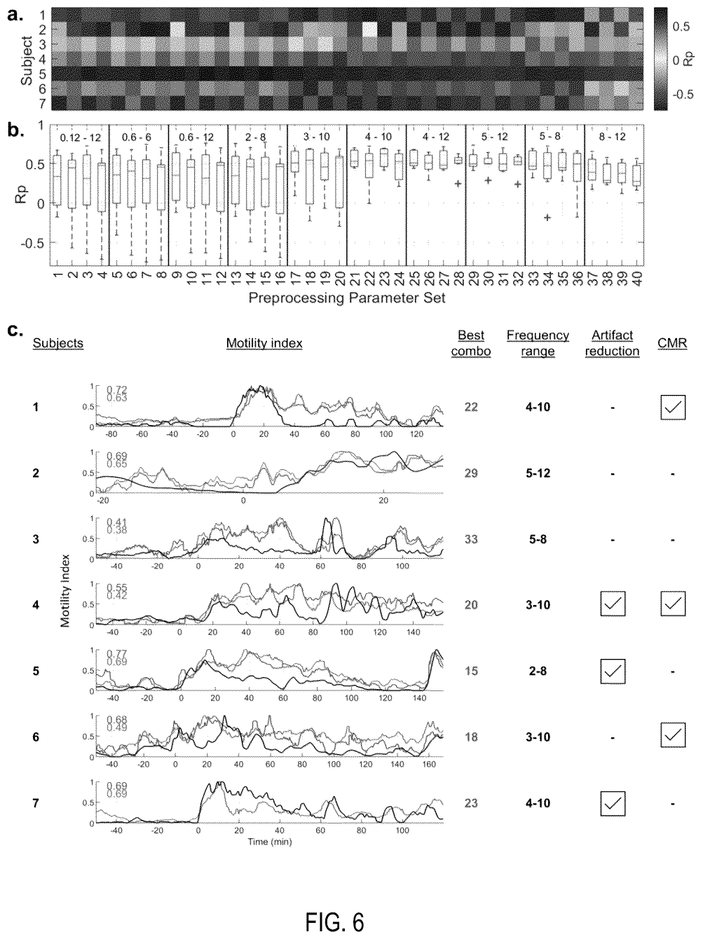

FIG. 6 illustrates motility index temporal correlation grid and time course of the best matching signal processing pipelines: (a) BSCM-HRCM MI correlation values across 40 different combinations (4 columns per frequency bandwidth tested) of signal processing performed for each of 7 subjects. The sets of 4 preprocessing filter combinations within a specified frequency band are specified as (artifact reduction, CMR)={(off, off); (off, on); (on, off); (on, on)}. Pseudocolor indicates value of Pearson correlation coefficient (Rp). (b) Box plot statistical summary computed across all subjects for each of 40 individual preprocessing parameter sets. Combination 23 (frequency 4-10 cpm, artifact reduction on, CMR off) yielded the cohort-wide best overall performance. (c) Motility index vs. time: black trace=HRCM MI, blue trace=maximum correlating BSCM MI; and red-orange trace=cohort-wide best overall preprocessing parameter set (number 23). Meal times are aligned at t=0 min. Every subject had a different preprocessing parameter set achieving maximal correlation, but there was a strong trend toward higher frequency range filtering achieving better overall performance.

### Statistical Analysis

Student's t-test was performed to compare the difference between the meal response outcomes measured from the HRCM and BSCM data. The null hypothesis was that there was no difference between the two datasets. Pearson's correlation coefficients were used to assess the strength of correlations between the motility index (MI) outcomes of the recordings. A p-value of <0.05 was deemed to show statistical significance between two datasets. Pearson coefficient values>0.5 were considered to indicate ‘good’ or substantial agreement between MI traces. GraphPad Prism version 9.5.1 (GraphPad Software, San Diego, California USA) and MATLAB R2022b (MathWorks, Natick, Massachusetts, USA) were used for statistical analyses and production of figures.

### Participants Information

A total of 11 participants were recruited with a median age of 50 (range 30-69) and majority were female (9:2). Simultaneous recordings for analysis were successfully achieved in 7 participants whose median BMI was 25.6 (range 22.3-31.3). Recordings from 4 subjects were excluded from further analysis due to: insufficient overlapping data due to a delayed X-ray and loss of over half of the manometry data due to setting/connection error (1), manometer insertion failure (1), manometer technical failure and inadequate BSCM data (1), and participant early withdrawal (1). One participant had a pre-arranged propofol anesthetic and one participant declined both sedation and analgesia. Indications for colonoscopies were family history (3), anemia (2) and mild GI symptoms at the time of referral which had settled completely by the time of investigation (2). All other participants received a combination of intravenous midazolam and fentanyl, and none were administered hyoscine or other antispasmodic medications (see Table 1). The median recording duration was 211 min (range 50-239). The first two of the analyzed cases had irregular pre-meal periods of 23 and 102 min due to being called for the X-ray imaging. In the subsequent studies, X-ray imaging was performed pre-recording, facilitating uninterrupted synchronous recordings of pre-meal (60 min) and post-meal periods.

TABLE 1
Demographics of participants

| Subject | | | | | | |
|---|---|---|---|---|---|---|
| ID | Age | Sex | Ethnicity | BMI | Clinical Indication | Sedation |
| 1 | 38 | F | Middle | 22.7 | Iron deficiency anemia and rectal | Propofol |
| | | | Eastern | | outlet bleeding | |
| 2 | 50 | M | NZ | 25.6 | Intermittent abdominal discomfort | Midazolam + |
| | | | European | | and dyspepsia | Fentanyl |
| 3 | 63 | F | NZ | 24.4 | Family history of bowel cancer and | Midazolam + |
| | | | European | | previous polyps | Fentanyl |
| 4 | 62 | F | NZ | 22.3 | Altered bowel habit and family | Midazolam + |
| | | | European | | history of bowel cancer | Fentanyl |
| 5 | 42 | F | Other | 25.8 | Bloating/epigastric pain | Midazolam + |
| | | | European | | | Fentanyl |
| 6 | 50 | F | NZ | 31.3 | Anemia | Midazolam + |
| | | | European | | | Fentanyl |
| 7 | 52 | M | NZ | 31 | Lynch syndrome - regular | Nil |
| | | | European | | surveillance | |

### Data Summary

Synchronously recorded BSCM and HRCM data from the 7 subjects with successful recordings were analyzed. A meal response occurred in all, with timings and duration observed to be highly concordant between HRCM and BSCM measurement modes. Temporal analysis of BSCM and HRCM motility indices demonstrated a high level of correlation (median Pearson r=0.69; 0.47-0.77) using the subject-specific optimal signal processing method for each case. Collectively across the cohort, the frequency bandwidth range 4-10 cycles per minute (CMR=off; LMMSE artifact reduction=on) was identified as the best single signal processing method (out of 40 possible) to achieve the highest correlation to the manometry data (median Pearson r=0.63; 0.43-0.69). Spatial analyses showed good agreement of the regions of CMP activation during the active and quiescent epochs. Participants' experience was significantly more positive with BSCM compared with HRCM; all participants unanimously preferred BSCM to HRCM and less discomfort was reported with BSCM (HRCM:median 7.5/10; range 2-9 vs. BSCM:median of 1/10; range 1-5; p=0.0005).

### HRCM Frequency and Best Correlating BSCM Frequency Bandwidths

Colonic intrinsic frequency was dynamic and generally rose to higher frequencies following the meal. For example, during subject 4's meal response CMPs were mainly above 6 cpm and showed 2-3 min bursts of high frequency activity in the rectosigmoid region up to 12 cpm. Subjects 3, 6 and 7 also had dynamic rises in their CMP frequency during the post meal period. Only subjects 1, 2 and 5 continued to exhibit only low frequency (˜2-4 cpm) CMPs in their post meal periods. These findings are all within the ranges of normal CMP physiology stated in the literature. Low frequency CMPs (2-4 cpm) were the dominant intrinsic frequency type (2-4 cpm activity: 46.4%, 4-10 cpm activity: 33.3%; p=0.036); however, the sequential frequency range analysis showed the opposite result; a significantly higher proportion of CMP activity incurred within the higher frequency bandwidth (4-10 cpm activity: 47.0% vs 2-4 cpm activity: 26.5%; p=0.007). The cohort-wide optimally correlating preprocessing settings were determined to be: frequency range=4-10 cpm, LMMSE=on; CMR=off.

### Motility Index Correlation

Overall, the CMP activity for every subject was successfully identified and correlated well between manometry and body surface measurement modes. Even highly dynamic motility was observed to be highly correlated. For example, subjects 5, 8, 9 and 11 all had secondary activities (rise in CMP activity after the primary meal response had completed per this study's definition of meal response) that were closely concordant in BSCM and HRCM MI traces vs time. The optimal (maximal correlation) signal processing parameter set varied across subjects. Artifact reduction was favorable in 3 out of 7 cases, similarly CMR results were just as divided. While searching for the best overall signal processing combination, out of the possible 40, the higher frequency filter bands, with the lower cutoff≥4 cpm, were significantly better in 4 out of 7 cases. Frequency bands with lower cutoffs≤3 cpm resulted in poor performance (negative correlation values) for 2 subjects (2 and 3), and ≤2 cpm cutoff additionally yielded reduced correlation for 2 more subjects (4 and 6). Across the 7 recordings, the signal processing combination with frequency bandwidth 4-10 cpm; artifact reduction on, CMR off offered the best cohort-wide performance (Pearson r mean±std=0.56±0.13; median=0.63; range=0.38-0.69). The motility index correlation using individually optimal signal processing pipelines led to modestly higher levels of correlation (Pearson r mean±std=0.64±0.12; median=0.69; range=0.41-0.77).

### Meal Response Match

Meal responses were successfully identified in all 7 subjects. All measured endpoints correlated strongly between manometry and body surface data. The meal response start time had a very strong correlation (Pearson r=0.998; p<0.0001). The meal response end time (Pearson r=0.83, p=0.041) and meal response duration (Pearson r=0.85; p=0.03) between HRCM and BSCM were also strongly correlated. Subject 2 was not included in the end time and duration analyses as the experiment ended early in the initial period of the meal response (53 min total recording duration). Meal response activities started at the beginning of the meal ingestion period, aside from subject 4, whose primary meal related activity rose just after the meal was completed. The correlation for meal response end time and duration correlation was weakened by subject 1, an outlier. At the end of subject 1's meal response on manometry, BSCM also shows a drop in the MI (approximately halved), however, the MI level is maintained above the baseline for another hour. The BSCM spatial analysis of subject 1 suggests that the meal response continues in a region not reached by manometry (in the proximal/right colon) which may account for the discrepancy between HRCM and BSCM.

Activity lines were generated by establishing a MI level threshold (2.5 standard deviations above pre-meal MI levels) above which the colon is deemed to be active as per previous work. ‘Similarity scores’ quantified the proportion of time in agreement over the course of the study (0—no agreement; 100=perfect agreement across the whole study period). For the ‘best’ BSCM trace (blue BSCM vs black HRCM activity lines) the similarity score was 78.0±4.8 (median=80.2. range=69.1-82.4). For the cohort-wide best analysis combination (red BSCM vs black HRCM activity lines) the similarity score was 78.2±8.0 (median=80.2; range=61.7-92.8). Thus, the activity line analysis, an objective measure of active times, demonstrated an overall high concordance, further corroborating the high degree of synchronicity in HRCM vs. BSCM meal response timings based on subjective visual identification via expert manual review.

### Spatial Analysis and Spatial Correlation

Spatial maps presented were made using the data resulting from the signal processing combination, number 23. This was done to test the sensitivity of the ‘best overall method’ to localize CMP hot zones and remove the impact that a CMR filter may have on creating accurate heat maps. Summary figures were produced with ‘main phases’ as defined from analysis of the HRCM data. These were pre-meal and meal response periods, which were ubiquitously present, as well as quiescent and secondary activity periods. Heatmap representation of CMP activity in different phases demonstrated spatio-temporal agreement between HRCM and BSCM. For example, two recordings (subjects 5 and 6) show that when CMP activity level is at baseline on HRCM (pre-meal and quiescent periods) the BSCM heatmap exhibits a concordant general paucity of activity. When the CMP activity levels are higher in the HRCM maps during the primary meal response and the secondary activity periods, the BSCM heatmaps show a similar dynamic rise in activity. A closer examination of the BSCM heatmaps revealed two main visual signatures of CMP activity; (1) bright foci on the array that represent dominant CMP active sources that lie under the array, i.e. distal colon/RSJ (c.f. secondary activity epochs of subjects 5 and 6); and (2) diffuse, low intensity changes across a large region of the body surface map caused by CMP activities projecting from a distance onto the BSCM array (c.f. proximal colon dominant primary meal response of subject 5). As the array covers the abdomen and the colon only in part, CMPs that arise from outside the array perimeter lose signal intensity inverse to the square of the source-sensor distance. The diffuse activity observed on the array can be attributed to the spatial volume conductor effect. In periods of multifocal activities, a mix of diffuse and focal activities were appreciable on the BSCM heatmaps making localization of specific hotspots challenging (e.g., during the primary meal response periods of subjects 2, 5, 6 and 7). Data was also analyzed in 10-min epochs to examine the heatmaps in finer temporal detail however, the main spatial analysis discussions presented in this study are based on the main phases.

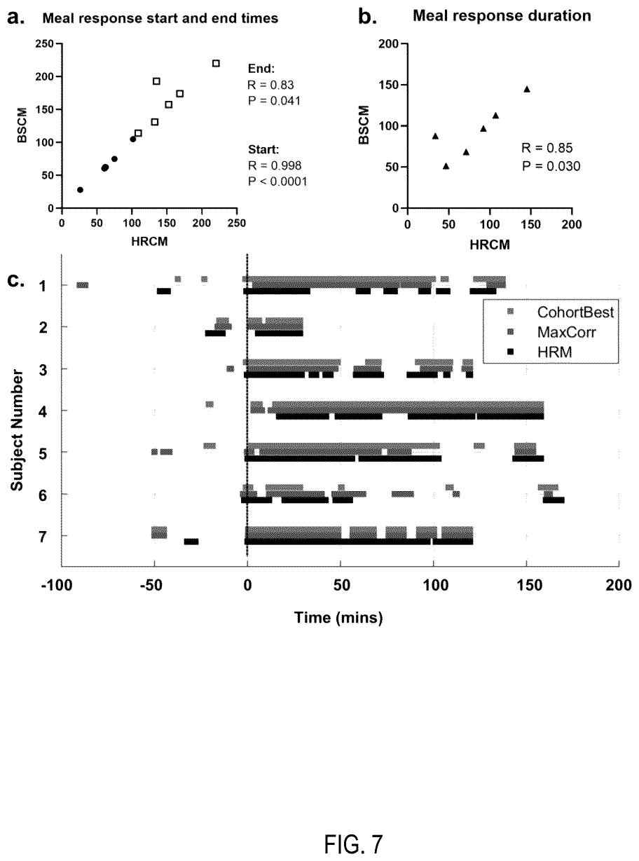

FIG. 7 illustrates a summary of CMP meal response match between HRCM and BSCM. FIG. 7 illustrates (a) High level of correlation observed for both start and end times of the meal responses; (b) Meal response duration (meal response end time-start time) was also well correlated. Values are in units of minutes. (c) Suprathreshold Activity Lines show a high level of agreement in active vs quiescent periods for all subjects. Black trace=HRCM; Blue=highest correlation BSCM; Red=cohort-wide optimum parameter combination BSCM trace. Meal times are aligned at t=0 min.

### Detection of Dynamic Shifts in the Regions of Dominant CMP Activity on BSCM

Cases 5 and 6 also display diachronous changes to the dominant pacemaker region which altered between different phases of the recordings. This provided an opportunity to assess the sensitivity of BSCM demonstrating the changes spatially. During subject 5's meal response period, the proximal colon is dominantly active, followed by a 35-min period of quiescence. In the final 30 min, the main activity is focused in the RSJ region. BSCM heat-maps are clearly in agreement with the manometry findings; during the meal response period, a diffuse activity pattern is observed across the array, but when the distal colon is dominant in the last 30 min, there is a focally bright spot localizing the CMP source to be directly over the RSJ area. In subject 6, the HRCM MI during the meal response is mainly driven by the distal colon and the right/proximal transverse. Here, two clear active foci are observed, one hotspot on the upper left (anatomical right) array, corresponding to the proximal colon's activity and another in the left right (anatomical left) corner that correlates to the RSJ/sigmoid regional activity. Subject 6's transverse colon hangs low, as opposed to subject 5's, thus the proximal colon's activity projects a brighter distinct focus onto the array. In the last 20 min of the recording, the transverse colon (directly above the array) is inactive while the distal colon remains strongly active, thus the singularly active distal colon region of activity is more clearly appreciable on the BSCM heatmap.

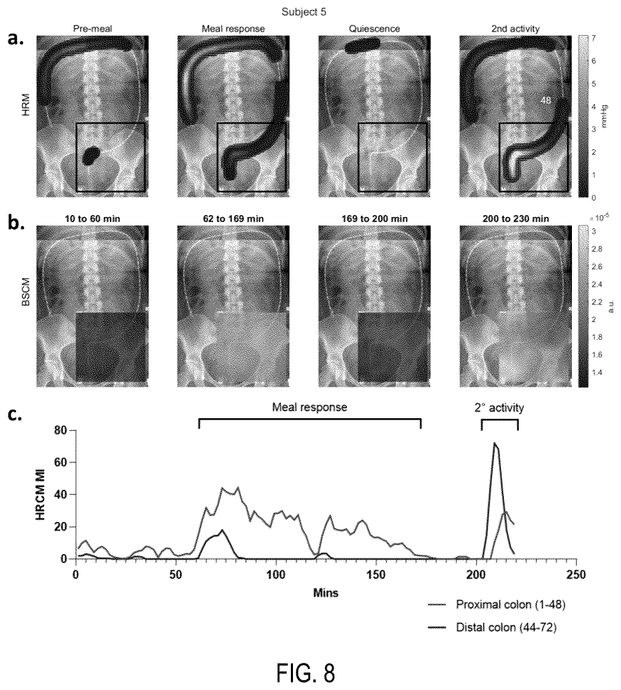

FIG. 8 illustrates spatial analysis of subject 5 with regional analysis of HRCM MI. Two key observations from HRCM spatial analysis (a) are the dominant right and transverse colon activity in the primary meal response (62-169 min) followed by an RSJ dominant secondary period of CMP activity (200-230 min). The shift in regional dominance is mirrored by BSCM (b). Distant dominant source (right/transverse colon) in the meal response period projects onto the array as a diffuse activation with weak hotspots over the RSJ and sigmoid colon. The RSJ activity in the last 20 min is strongly represented by a corresponding hotspot on the lower (anatomical) right corner of the array. The corresponding HRCM MI graph (c) shows the clear shift in regional dominance of CMP activity. The sensor numbers 44-48 overlap for proximal and distal colon, but this was to allow for a few CMPs crossing over into the adjacent regions; no CMPs were double counted.

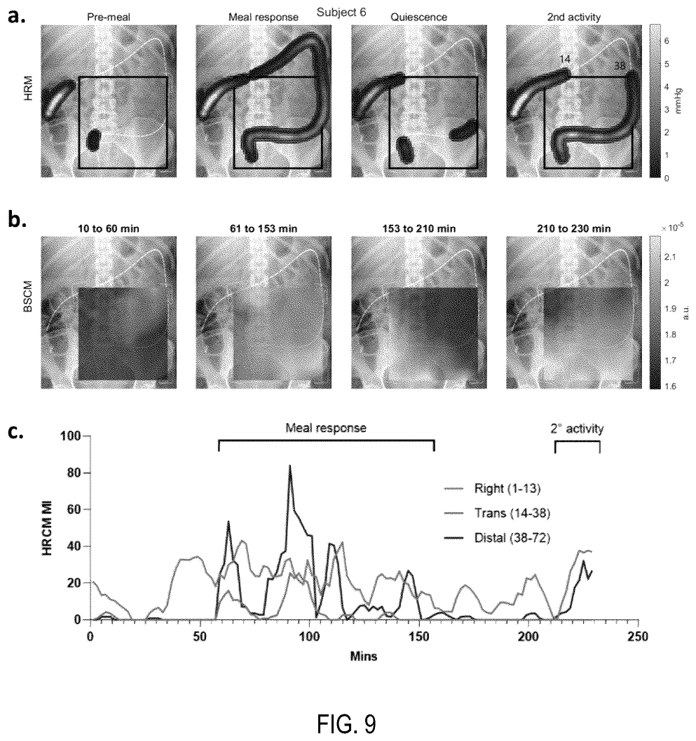

FIG. 9 illustrates spatial analysis of subject 6 with regional breakdown of HRCM MI. Subject 6 had a multifocal meal response involving most of the colon (a). The transverse colon is closer to the BSCM array, so its activity projects a more focal impression on the top (anatomical) right side of the array during the meal response period (as seen on row b). During the second active period (i.e. the last 20 min), the distal transverse colon (sensors 14-38) exhibit no CMP activity (as seen on rows a and c) and the intensity at the top of the BSCM array is concordantly minimal.

### Participant Survey

All participants preferred BSCM to HRCM. All participants who underwent both concurrently commented that BSCM was less invasive, easier, and quicker. Significantly less discomfort was incurred with BSCM (median 6.5/10 with HRCM vs. 1/10 with BSCM; p=0.0005). Only one participant mentioned that removal of the BSCM array caused discomfort as they had sensitive skin but would still prefer BSCM over HRCM. Four participants added that not requiring bowel prep was an important factor in choosing BSCM to be their preferred choice of investigation. Two participants expressed that the potential for BSCM to be undertaken out of the hospital setting without sedation and causing less disruption to work life (due to bowel prep and hospital day admission) were potential advantages.

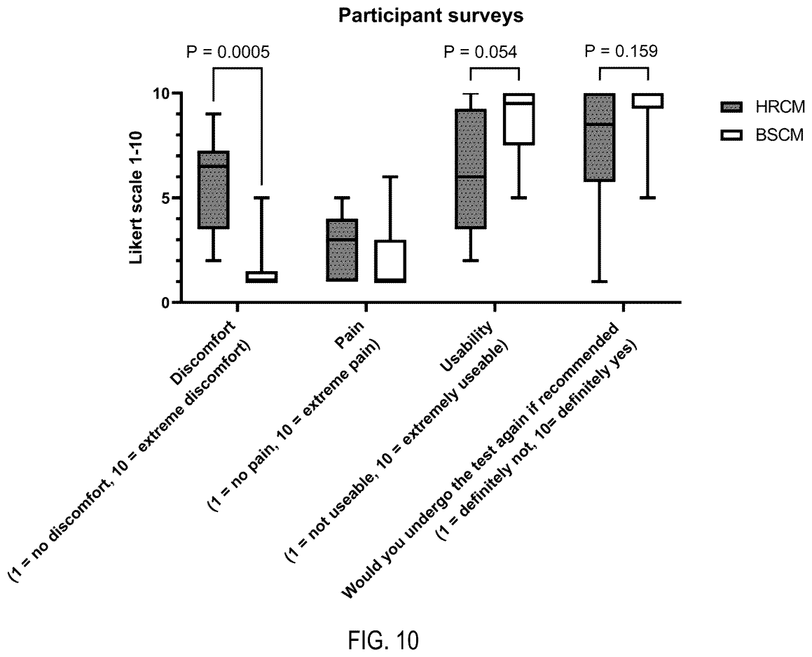

FIG. 10 illustrates a box plot summary of the participants' numerically reported outcomes. Non-invasive BSCM was found to be significantly more comfortable than HRCM. BSCM was also favorable in terms of usability and acquiescence to repeated testing. Neither investigation inflicted significant levels of pain on the participants.

### Discussion

This study has validated BSCM as a non-invasive technique against HRCM for detecting colonic CMP activity from body surface electrical recordings. The results show that BSCM can detect CMP activity in the colon, quantify meal responses, and spatially locate hotspots of CMP activity with a high level of correlation to HRCM. BSCM's significantly updated CWT-based signal processing pipeline, informed from a rich set of colonic physiology analyses via HRCM, was crucial for performing temporal correlation and spatial map analyses. The study presents clear evidence that BSCM is sensitive and specific to colonic CMPs, exhibiting a high degree of spatiotemporal correlation with HRCM analyses.

As HRCM and BSCM are fundamentally different measurement modalities, motility indices were used for quantitative comparison. Motility index has been described by a number of previous manometry studies to characterize the activity density in a given epoch using an area under the curve (AUC) approach, the definition of which has not been standardized for interpreting high resolution data. In the current study, HRCM MI was defined as a product of the 3 key HRCM outputs (number of CMPs, mean amplitude, and propagation length). The current HRCM MI metric cannot differentiate the CMP activities of lower frequency (number of CMPs per unit time) with longer distances vs. higher frequency with shorter lengths in an analysis window. The BSCM MI correlation should be better in the former case but may be mismatched in the latter. An important limitation of the BSCM technique is that the directions of wave propagation cannot yet reliably be ascertained due to the complex and variable anatomy of the colon. Traditionally, HRCM studies have reported a mixture of ante- and retrograde CMP activities, with directionality likely to play a key role in storage and organization of bowel contents, controlling passage of contents to the rectum, and the maintenance of continence.

The bandpass filter frequency range selected for the BSCM analysis method was found to be the single most significant parameter for identifying the colonic electrophysiological signal referenced to HRCM. There was only one case (subject 5) for which correlation improved slightly with a lower frequency range approach (2-8 cpm), likely owing to the fact that a single region/single pacemaker was predominantly active at any one time, with an intrinsic frequency range of 2-4 cpm. It is worth noting that the high frequency analyses also performed well for this case. The overall superior performance of the 4-10 cpm bandwidth may be attributed to the following three factors. First, the high intrinsic frequencies in this range were observed in some cases (notably in subjects 3, 4 and 7). Further analysis of HRCM frequency distributions revealed that the largest post-vs pre-meal distribution changes occur in frequency bands >3 cpm. Second, the superposition of multiple CMP sources generates multiphasic waveforms manifesting as higher frequencies. For instance, independent colonic sources generating CMPs in the 2-6 cpm range, but out of phase by 10 s, may result in a strong >6 cpm frequency (i.e., sequential frequency). Lastly, higher frequency bandwidth screens out gastric slow wave activity most efficiently. To summarize, based on the current study's findings, the frequency range 4-10 cpm, artifact windowing, and no CMR (combination 23) was the most robust combination, and may provide a suitable analysis pipeline for future BSCM studies when HRCM referential data is absent. A caveat to the generalization of the study's results is that the effects of the unnatural conditioning of the colon prior to recording (bowel preparation and endoscopic insertion of the HRCM catheter) on CMP activity remain to be investigated.

It is also worth noting that the optimal colonic filter bandwidth substantially differs from the 0.6-6 cpm previously validated for detecting gastric activity using BSGM, which may be useful for dual colonic and gastric monitoring in the future. Whereas gastric slow waves usually propagate at a stable frequency with minimal variance (3.04 cpm; reference interval: 2.65-3.35 cpm), the colon's CMP frequency range is broader with a more intermittent/sporadic temporal activity profile. Another major difference is that the stomach normally has a single dominant pacemaker, but the colon has multiple independent and simultaneously active regions. While others have reported a colonic frequency range measured on the body surface of 12-20 cpm, frequencies up to a maximum of about 12 cpm from HRCM analysis have been observed, which is consistent with the majority of manometry studies. Also, the data of this study shows that the observed frequency range from the body surface is not always directly congruent with actual colonic frequency range (due to summation effects), yet it still remains below 12 cpm. Care must be taken to differentiate colonic CMPs vs. respiratory artifacts in the 12-20 cpm frequency band.

The spatial mapping of the case studies presented herein illustrate for the first time that focal regions of activity can be approximately localized using the BSCM techniques. When a singular dominant region of the colon is present two observations are made: activities occurring directly beneath the array appear as focal hotspots, and activities outside of the array's area are perceived as diffuse and broad low level of activity sensed across a large region of the array edge. The current study's analysis also indicates that simultaneous multifocal activities lead to more diffuse heat maps that make localization challenging. This issue could be mitigated with a larger area BSCM array that would overlay the entire colon; thus CMP hotspots may be visualized with higher accuracy and focus; however, the current array setup used in this study is already a significant manufacturing challenge. More studies in the future that display dominance of meal response in different, singular regions in the colon, would be instrumental for estimating the expected contribution from different colonic sources projecting onto the spatial map.

It is important to consider to what extent rhythmic electrical activity from other abdominal sources, namely the small bowel and urinary bladder, couple into the BSCM heatmaps. The small bowel operates at a frequency of about 12-15 cpm in the duodenum, decreasing along its length to 8-10 cpm in the ileum. The BSCM's optimal 4-10 cpm filter band is therefore expected to attenuate components arising from the proximal small bowel. The ileum frequency range does overlap; however, its contributions are expected to be relatively minimal based on the simple biophysical model that propagating waves would be represented by randomly oriented, small magnitude dipoles owing to adjacent and anti-parallel sections of tissue. The urinary bladder has been reported to operate at a frequency of about 2.2 cpm, thus the BSCM filter band set for 4-10 cpm is also expected to strongly attenuate its components as well. In summary, BSCM heatmaps feature contributions primarily from the colon and minimally, if any, from other abdominal sources.

A limitation of the current spatial mapping technique is the assumption that electrical activities would project directly anteriorly and interpret an inherently 3-D biophysical activity using a 2-D single X-ray image. The distortion in the heatmaps is expected to be minimal in the middle of the BSCM electrode array, which is co-planar with the X-ray, and may be more pronounced at the edges where the orientation deviates most from being coplanar, dependent upon the subject-specific abdominal radius of curvature. BSCM with cross-sectional imaging would likely improve accuracy in interpreting spatial patterns with a better appreciation of the variably convoluted course of the colonic tract and the abdominal wall's shape and depth, but poses logistical, cost, and radiation challenges.

In the current study, the whole array's data were used to derive the BSCM motility index which correlated well with manometry MI output from distal half to nearly full lengths of the colon. Many translational manometry studies have only quantified the CMP activities arising from the distal half of the colon/rectosigmoid region and it remains unclear what role proximal colon CMPs may play in function. Localization and analysis of specific regional activities are easier with manometry data by selecting only sensors that correlate either anatomically or physiologically with the colon region in question. However, regional analysis with BSCM is challenging due to several factors. First, the BSCM array only covers a limited segment of the colon directly, thus it is difficult to ascertain the exact source of electrical activities arising from outside the array covered area (i.e. proximal or transverse colon). In addition, due to the position of the ground and reference electrodes, on the right side (anatomical) of the array, the right colon's CMP activity injects a common mode signal into the rest of the measurement electrodes with variable intensity depending on distance from the active current sources. In some cases (subjects 4 and 6) CMR appeared to be beneficial in correcting a predominant activity pattern observed via HRCM in the ascending colon, beneath the ground and reference electrode. However, application of CMR worsened the correlation in 2 cases where simultaneous multifocal activity was observed (subjects 5 and 7).

One limitation of this study is the small cohort size, which reflects the highly technical and challenging experimental technique which also poses difficulties to patient recruitment and throughput owing to the invasiveness of manometry, procedural stressors, and time factors, as well as COVID-related mandatory restrictions during the study period. However, together the data set is rich in that every case provided sufficient physiological and anatomical variations (frequency, regional activities, colonic anatomy, and manometer insertion depth) to inform an analysis pipeline of strong correlation which could be applied in future BSCM studies.

Although HRCM served as the ground truth in this study, it must be recognized that HRCM has a limited scope based on the extent to which the catheter is inserted (the manometer could not reach the right colon in 3 out of 7 cases). In contrast, BSCM electrodes detect a superposition of all activities, with weighted intensities dependent on source-sensor distances. For example, subject 1 had less than half the colon's length measured by the manometer resulting in the biggest discrepancy in meal response correlations to BSCM. The data from the full colon manometry studies show that the proximal colon invariably switches on during the meal response period and BSCM heatmap analysis of subject 1 also suggests that the proximal colon remains active during the quiescent period.

In summary, this study has demonstrated three different metrics with validation that the resulting signal source from BSCM analysis is reliably of colonic origin: (1) motility index correlations, (2) meal response synchronicity, and (3) spatial hotspot analysis. CMP hypoactivity or hyperactivity has been associated with the development of low anterior resection syndrome (LARS) 17, fecal incontinence, postoperative ileus, and irritable bowel syndrome (IBS), constipation, such that these newly validated BSCM biomarkers could serve to guide clinical therapies. This is particularly significant because while HRCM remains an important research tool, it is not widely available, relatively invasive, and its data is time-consuming and complex to analyze. BSCM, on the other hand, may be performed without anesthesia nor endoscopy, and interrogates the colon in its physiologically natural/unprepped state for longer durations. BSCM, as validated in this study, therefore has significant potential to generate a meaningful mechanistic understanding of functional disorders and guide clinical therapies.

Referring now to high amplitude propagated contractions (HAPCs), HAPCs cause mass movements toward the distal part of the rectum and defecation. There is a number of disorders that HAPCs play a role in such as various forms of IBS. For example, a patient experiencing IBS-D may be affected by one or more motilities where, either contractions are moving too fast or too strongly such that processes are occurring too fast, or there is a delay in cyclic motor patterns that act as a brake to stop everything downstream. Constipation may be similarly caused by contractions that do not occur or occur in the wrong direction. Unlike electrical data associated with the stomach, which is relatively rhythmic, colonic electrical data often appears as bursts that may be confused with movement artifacts or the like. Various embodiments of the present disclosure enable analysis of HAPCs using BSCM electrical signals gathered using embodiments described herein.

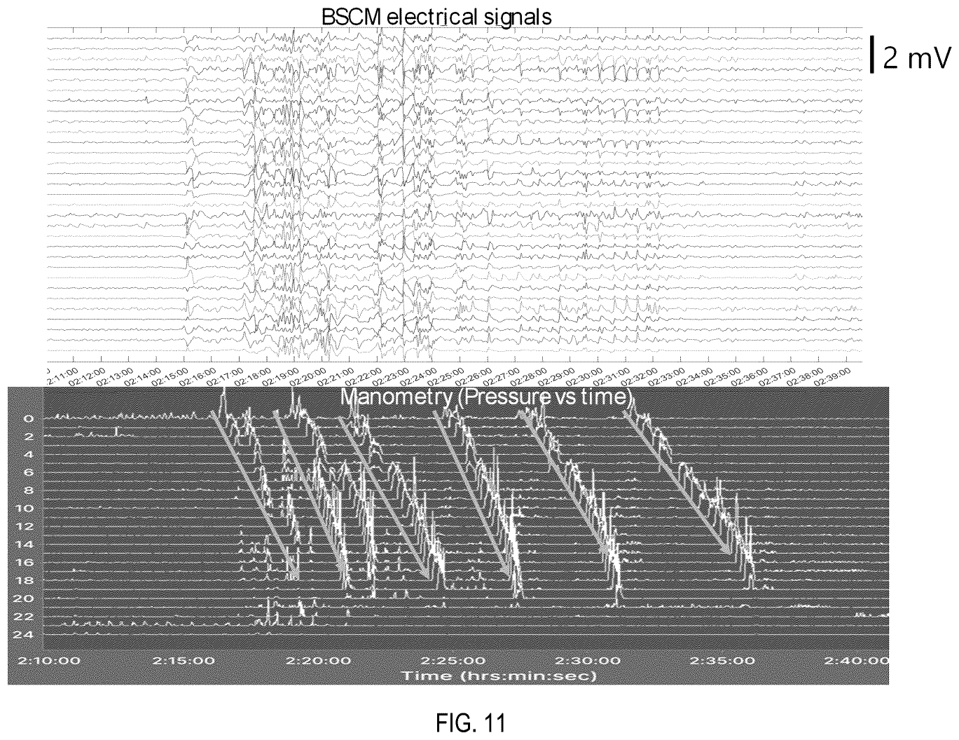

FIG. 11 illustrates an array of BSCM electrical signals correlated with manometry readings showing non-invasive detection of high-amplitude propagating contractions. Contemporaneous BSCM electrical signals and manometry readings may be gathered to validate the temporal correlation of the BSCM electrical data to ground truth manometry data as indicated by the arrows. Strong concordance is shown between the BSCM electrical data and the manometry data. Concordance is assessed by time-series correlation of BSCM MI to HRM MI (e.g., the ground truth). In an exemplary embodiment, for a N=4 validation, the Pearson correlation coefficient is computed to compare: MI across the entire study where (corr coeff median=0.62; mean+/−std=0.61+/−0.06; range=0.52-0.65) and MI limited in time to when HAPCs occur where (corr coeff median=0.71; mean+/−std=0.71+/−0.13; range=0.56-0.85).

According to various embodiments, a plurality of electrodes are represented in the BSCM electrical data above the manometry data. For example, each channel may represent an electrode on the BSCM electrode patch. One or more electrodes, and associated electrode data, may be removed from the validation analysis in response to determining that the connection was not complete, the data was too noisy, etc.

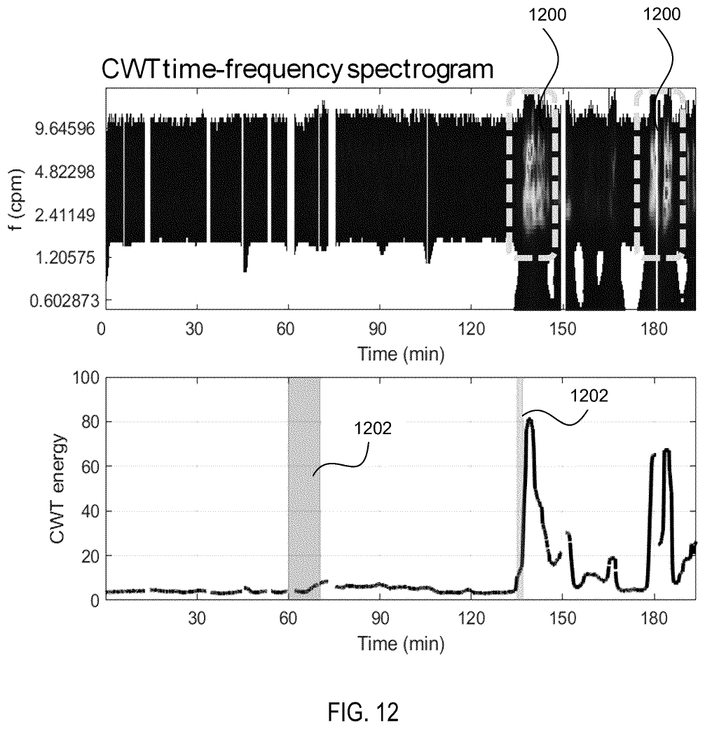

FIG. 12 illustrates BSCM electrical signals including exemplary HAPCs signatures 1200. HAPCs signatures are illustrated in the time domain (e.g., the top portion) and the time-frequency domain (bottom portion). In the time domain, HAPCs may be characterized by amplitudes of >200 uV that persist for at least 1 minute. In the time-frequency-domain, HAPCs may be characterized by activity predominant in the frequency band of approximately 2-12 cpm, and often occur in 2 distinct bands (2-4 cpm vs 8-12 cpm). Once the BSCM electrical signals are validated as appropriately representing colonic electrical activity, the BSCM electrical signals may be further assessed and evaluated for determining HAPCs signatures, biomarkers, other normalized biometrics, etc. The bottom portion of FIG. 12 illustrates the amplitudes corresponding to the data above and illustrates the sharp increases in amplitude corresponding to the HAPCs signatures 1200. Various stimuli 1202 may be provided to the patient to further validate and/or correlate the data to various biomarkers. For example, the stimuli 1202 may include eating a meal, introduction of a medication, or the like, that drives colonic activity.

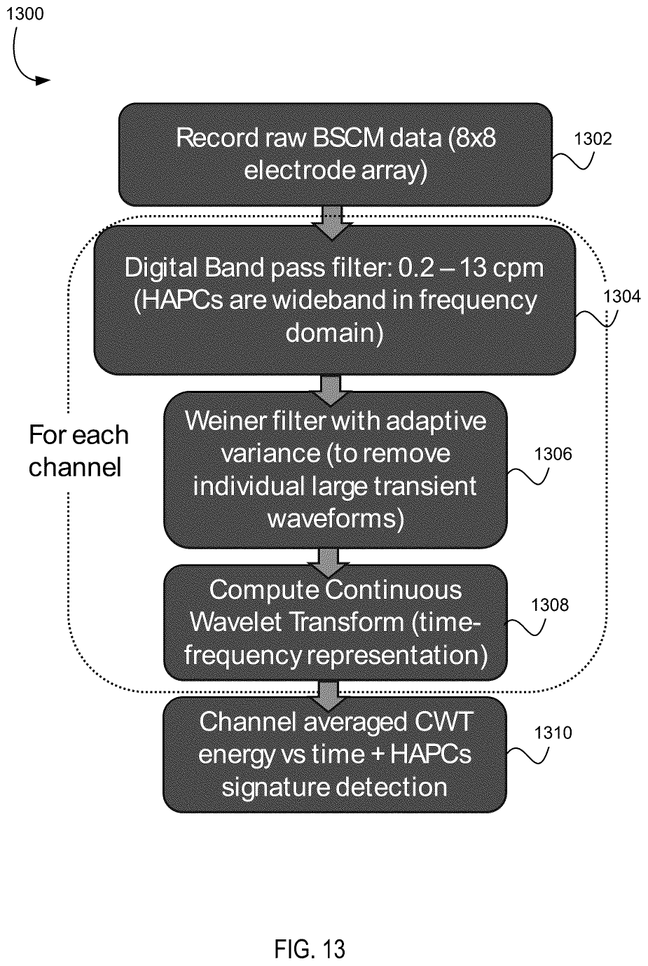

FIG. 13 illustrates a flowchart for assessing BSCM electrical signals for HAPCs detection. Process 1300 illustrates multiple steps for assessing the raw data (e.g., the BSCM electrical signals) to identify HAPCs signatures. Box 1302 includes recording raw BSCM data from the electrode patch array (e.g., 8×8 electrode array) as described in various embodiments of the present disclosure (e.g., FIGS. 1-3). For each channel of data (e.g., collected from each individual electrode), the steps of box 1304, box 1306, and box 1308 are performed. Box 1304 includes applying a digital band pass filter to the raw data, for example a filter in a range from 0.2-13 cpm as HAPCs are wideband in frequency domain. For example, any electrical signals determined to be outside a predetermined threshold frequency bandwidth are removed from the data. In various embodiments, this threshold is adaptive. An adaptive threshold is particularly advantageous due to the burst-type signals generated by HAPCs. For example, data may be evaluated based on a subset of data, rather than the full set of data and the threshold may be adjusted accordingly, based on at least some embodiments.

Box 1306 may include applying a Weiner filter with adaptive variance for removing individual large transient waveforms. For example, box 1306 may include removing data determined to be associated with movement artifacts or the like. In some embodiments, box 1306 further includes evaluating how much variance there is in the data (e.g., how much noise there may be and how differentiated it is from the rest of the data). Box 1308 includes computing a continuous wavelet transform (e.g., a time-frequency representation) to determine what frequencies were present at what time during the data. Box 1310 includes averaging the channel CWT energy vs. time with the HAPCs signature detection to validate any HAPCs biomarkers to be used in diagnostic applications.

BSCM and HRCM data motility indices (MI) are computed using separate processing pipelines. The backbone of both pipelines is a time-frequency representation computed using the Continuous Wavelet Transform (CWT). The outputs of each produce and time series which are then quantitatively compared via Pearson correlation coefficient.

In summary, according to various embodiments, the time correlation algorithm for the HRCM data includes, for each channel, the CWT is computed. The fundamental frequency of individual pressure events occurring during HAPCs is isolated in the CWT spectrogram by reducing harmonics. The CWT is averaged for all sensors to produce a ‘global’ time-frequency representation for manometry. The CWT coefficient magnitudes are summed at each moment in time within the 0.2-2 cpm frequency band, which is the dominant frequency of HAPCs.

In various embodiments, for the BSCM, for each channel, a digital bandpass filter (0.2-13 cpm) is applied to the electrical signal. A Wiener filter with adaptive variance noise parameter is applied to remove excessively large amplitude waveforms which correspond to motion artifacts. The Wiener filter compares the local variance (30 s windows) of the waveform to the estimated noise level. The estimated noise level may be calculated in a sliding (10 min width) time window and may update in time over the duration of the experiment. Accordingly, episodic colonic motility corresponding to electrical signals which vary in time, may be seen by the algorithm as signal, not noise. The CWT is computed for each channel. Acceleration vs time data recorded by the electrode array patch connector device may be processed to find time points at which large accelerations occur in a statistical sense (acceleration exceeds a threshold computed as the median acceleration+a multiple of the median of the absolute deviation). The timepoints exceeding these thresholds may be presumed to motion artifact corrupted. The CWT may be blanked (coefficient values set to 0) according to the e-folding time of the mother wavelet, centred on the marked timepoint. The mean CWT coefficient magnitudes may be computed across all channels to yield a ‘global’ time-frequency representation of the BSCM recordings. The BSCM MI is computed by computing by summing at each point in the time the coefficient values of the global CWT spectrum.

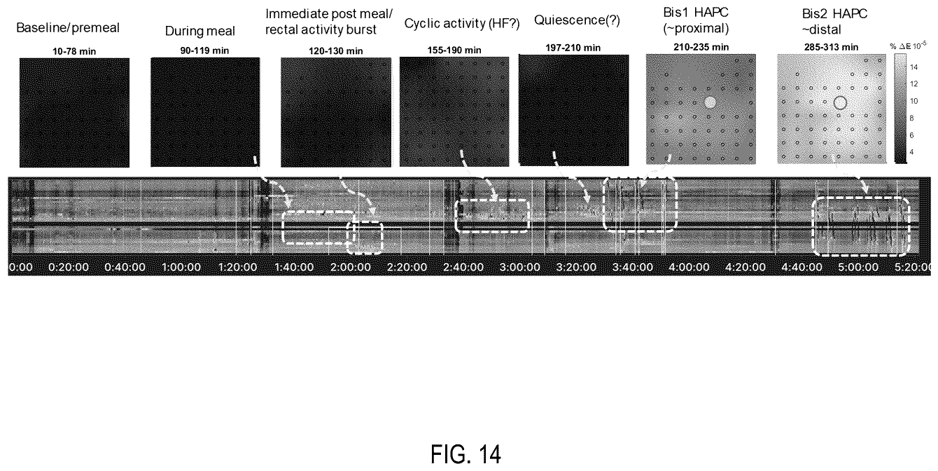

FIG. 14 illustrates spatial mapping of BSCM electrical signals. Each portion of FIG. 14 illustrates a visual representation of the electrical signals at various times. For example, the BSCM may be used to generate spatial maps over time indicating the ‘hotspots’ of activity, thus localizing GI electrical sources (e.g., active segments of the colon).

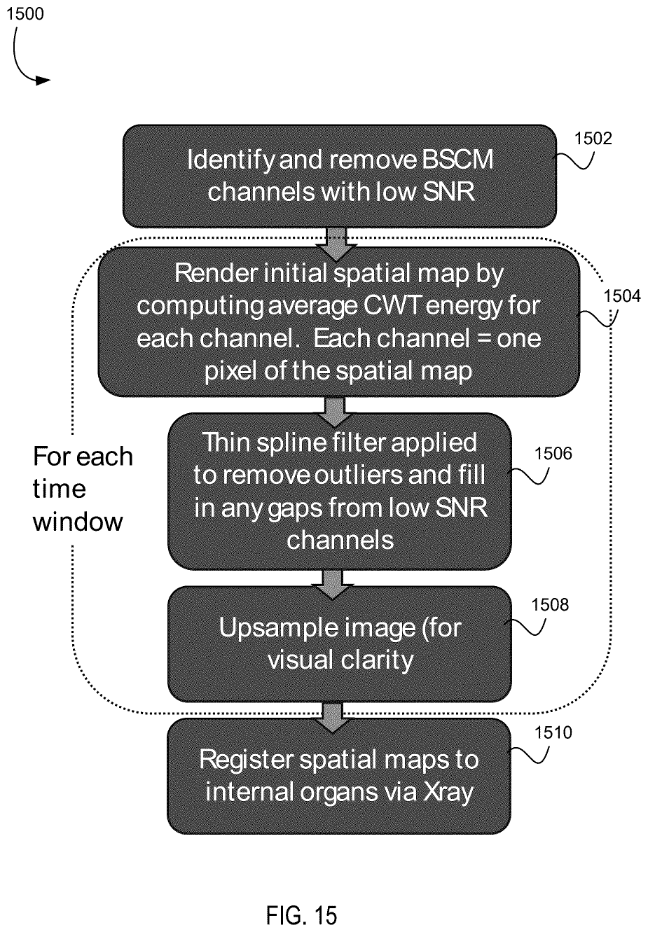

FIG. 15 illustrates a flowchart for spatially mapping BSCM electrical signals. to localize sources of electrical activity over time, thus identifying active segments of colon. The input to this algorithm is the BSCM output from process 1300 described above. For example, process 1500 describes a method for producing the spatial maps as shown in FIG. 14. Box 1502 includes identifying and removing BSCM channels with a low SNR. For example, any electrodes that did not collect good quality data (e.g., noise below a predetermined threshold), may be removed from the data. In some embodiments, the criterion for bad channel detection includes one or more of the following: impedance is a threshold value (500 kohm); amplitude is a statistical outlier (very low amplitude=‘dead channel’; very high amplitude=‘disconnected channel’); frequency spectrum is dissimilar from other nearby channels (<0.3 correlation). For each channel of predetermined time window, box 1504, box 1506, and box 1508 are performed. Box 1504 may include rendering an initial spatial map by computing the average CWT energy for each channel. In some embodiments, every channel represents at least one pixel on the initial map. For example, the 8×8 initial spatial map is rendered by averaging the CWT coefficient values within a given time window (typically 10 min). Box 1506 may include applying a thin spline filter to remove any remaining outliers and fill in any gaps from the low SNR channels that were removed. Box 1508 may include upsampling the image for visual clarity. In some embodiments, step 1508 may include upsampling the 8×8 grid image by a factor of 10× for visual clarity. Box 1508 may include registering the spatial maps to internal organs via an X-ray or fluoroscopy images or the like.

While exemplary embodiments have been illustrated and described, it will be appreciated that various changes can be made therein without departing from the spirit and scope of the disclosure.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present, or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning, allowing for inclusion of not only the listed components or elements, but also other non-specified components or elements. The terms ‘comprises’ or ‘comprised’ or ‘comprising’ have a similar meaning when used in relation to the system or to one or more steps in a method or process.

As used hereinbefore and hereinafter, the term “and/or” means “and” or “or”, or both. As used hereinbefore and hereinafter, “(s)” following a noun means the plural and/or singular forms of the noun. As used hereinbefore and hereinafter, the term “continuous” or “semi-continuous” with respect to the test period is to be interpreted as ongoing throughout the entire or nearly entire test period.

In some examples, values, procedures, or apparatuses are referred to as “lowest”, “best”, “minimum,” or the like. It will be appreciated that such descriptions are intended to indicate that a selection among many used functional alternatives can be made, and such selections need not be better, smaller, or otherwise preferable to other selections.

What is claimed is:

1. A system for mapping colonic and rectal activity of a patient, the system comprising:
an electrode array patch having a plurality of electrodes configured to non-invasively measure electrical signals associated with colonic and rectal activity of the patient over a predetermined time period; and
a processor configured to:
receive the measured electrical signals from the electrode array patch over the predetermined time period;
determine one or more abnormal cyclic motor patterns (CMPs) based at least in part on the received measured electrical signals; and
generate a report comprising at least the determination of the one or more abnormal CMPs.

2. The system of claim 1, the processor being further configured to process the measured electrical signals using a continuous wavelet transform (CWT) analysis to characterize the measured electrical signals within a predetermined frequency bandwidth.

3. The system of claim 2, wherein the predetermined frequency bandwidth is between 4 and 10 cycles per minute.

4. The system of claim 2, the processor being further configured to compute a body surface colonic mapping (BSCM) motility index (MI) based on the CWT analysis.

5. The system of claim 4, wherein the BSCM MI comprises a CWTmaxval, wherein the CWTmaxval is a mean of a top 10% of wavelet coefficient values at one or more time points during the predetermined time period.

6. The system of claim 1, wherein the electrode array patch is disposed on the lower abdomen for measuring electrical signals arising from a rectosigmoid junction, an infraumbilical region, or a left colon.

7. The system of claim 1, wherein the determination of one or more abnormal CMPs correlates to a colonic motility disorder.

8. The system of claim 7, wherein the colonic motility disorder comprises fecal incontinence, constipation, irritable bowel syndrome, or low anterior resection syndrome.

9. The system of claim 1, wherein the predetermined time period comprises a meal response duration between 30 minutes and 180 minutes.

10. The system of claim 1, the processor being further configured to generate spatio-temporal mapping of the colonic and rectal activity.

11. A method for mapping colonic and rectal activity with an electrode array patch disposed over an abdomen skin surface of a patient, the method comprising:
non-invasively measuring electrical signals associated with colonic activity of the patient with the electrode array patch over a predetermined time period;
receiving the measured electrical signals from the electrode array patch over the predetermined time period;
determining one or more abnormal cyclic motor patterns (CMPs) and/or abnormal high amplitude propagated contractions (HAPCs) based at least in part on the received measured electrical signals; and
generating a report comprising at least the determination of the one or more abnormal CMPs and/or abnormal HAPCs.

12. The method of claim 11, wherein determining comprises simultaneously detecting CMPs and HAPCs.

13. The method of claim 12, wherein determining further comprises differentiating between CMPs and HAPCs.

14. The method of claim 11, wherein the report comprises a summation image of both the CMPs and HAPCs activity over time.

15. The method of claim 14, wherein the summation image comprises a far field and/or volume conduction.

16. The method of claim 11, wherein determining comprises independently detecting CMPs or HAPCs.

17. The method of claim 11, further comprising using a continuous wavelet transform (CWT) analysis to characterize the measured electrical signals within a predetermined frequency bandwidth for each of the CMPs or HAPCs.

18. The method of claim 17, wherein the predetermined frequency bandwidth is between 4 and 10 cycles per minute for CMPs.

19. The method of claim 17, wherein the predetermined frequency bandwidth is between 0.2 and 13 cycles per minute for HAPCs.

20. The method of claim 11, further comprising disposing the electrode array patch on the lower abdomen for measuring electrical signals arising from a rectosigmoid junction, an infraumbilical region, or a left colon.

21. The method of claim 11, wherein the determination of the one or more abnormal CMPs and/or HAPCs correlates to a colonic motility disorder.

22. The method of claim 21, wherein the colonic motility disorder comprises fecal incontinence, constipation, irritable bowel syndrome, or low anterior resection syndrome.

23. A system for mapping colonic and rectal activity of a patient, the system comprising:
an electrode array patch having a plurality of electrodes configured to non-invasively measure electrical signals associated with colonic and rectal activity of the patient over a predetermined time period; and
a processor configured to:
receive the measured electrical signals from the electrode array patch over the predetermined time period;
process the received measured electrical signals using a continuous wavelet transform (CWT) analysis;
compute a body surface colonic mapping (BSCM) motility index (MI) based on the CWT analysis to characterize the measured electrical signals as one or more abnormal high amplitude propagated contractions (HAPCs); and
generate a report comprising at least the characterization of the one or more abnormal HAPCs.

24. The system of claim 23, wherein using the CWT analysis to characterize the measured electrical signals is performed within a predetermined frequency bandwidth.

25. The system of claim 24, wherein the BSCM MI based on the CWT analysis is a sum of coefficient values of a CWT spectrum generated by the CWT analysis.

26. The system of claim 24, wherein the predetermined frequency bandwidth is between 0.2 and 13 cycles per minute.

27. The system of claim 23, the processor being further configured to apply a Weiner filter with adaptive variance for removing individual large transient waveforms.

28. The system of claim 23, wherein the electrode array patch is disposed on the lower abdomen for measuring electrical signals arising from a rectosigmoid junction, an infraumbilical region, or a left colon.

29. The system of claim 23, wherein the characterization of the one or more abnormal HAPCs correlates to a colonic motility disorder.

30. The system of claim 29, wherein the colonic motility disorder comprises fecal incontinence, constipation, irritable bowel syndrome, or low anterior resection syndrome.

31. The system of claim 23, wherein the predetermined time period comprises a meal response duration between 30 minutes and 180 minutes.

32. The system of claim 23, the processor being further configured to generate spatio-temporal mapping of the colonic and rectal activity.

33. A system for mapping colonic and rectal activity of a patient, the system comprising:
an electrode array patch having a plurality of electrodes configured to non-invasively measure electrical signals associated with colonic and rectal activity of the patient over a predetermined time period; and
a processor configured to:
receive the measured electrical signals from the electrode array patch over the predetermined time period;
determine one or more abnormal cyclic motor patterns (CMPs) based at least in part on the received measured electrical signals;
determine one or more abnormal high amplitude propagated contractions (HAPCs) based at least in part on the received measured electrical signals; and
generate a report comprising at least the determination of the one or more abnormal CMPs or abnormal HAPCs.

34. The system of claim 33, wherein the report comprises a summation image of both the CMPs and HAPCs activity over time.

35. The system of claim 34, wherein the summation image comprises a far field and volume conduction.
